(19) United States
(12) Patent Application Publication
(10) Pub. No.: US 2022/0062116 A1
(43) Pub. Date: Mar. 3, 2022

(54) Novel Expanding Copolymers

(71) Applicant: Polymer Competence Center Leoben GmbH, Leoben (AT)

(72) Inventors: Frank Wiesbrock, St. Michael in Obersteiermark (AT); Matthias Sebastian Windberger, Forchtenstein (AT)

(21) Appl. No.: 17/433,466

(22) PCT Filed: Mar. 8, 2019
(86) PCT No.: PCT/EP2019/055862

§ 371 Date: Aug. 24, 2021

(51) Int. Cl. A61K 6/891; C08G 73/02; C08G 64/02; C08G 64/30; C08G 64/40; C08J 3/24

(52) U.S. Cl. CPC A61K 6/891; C08G 73/0233; C08G 64/0241; C08G 64/302; C08G 64/40; C08J 2479/02; C08J 3/247; C08J 2369/00; C08J 2379/02; C08J 2469/00; C08J 3/244

(57) ABSTRACT

The present invention relates to expandable, polymerizable compositions comprising at least one benzoxazine and at least one cyclic carbonate, to polymerization products of these expandable, polymerizable compositions, to a process for preparing these polymerization products as well as to uses of these expandable, polymerizable compositions. The present invention is based on the surprising finding that copolymerizing benzoxazine monomers with cyclic carbonate monomers results in novel copolymers having unforeseeably high expansion rates, wherein the properties (e.g. solid/brittle, solid/soft, rubbery) of the resulting copolymers can be easily and reproducibly tuned/adjusted, depending on the ratio of the benzoxazine equivalents/cyclic carbonate equivalents present in the composition and copolymer, respectively.

The present invention relates to a novel expandable, polymerizable composition, a polymerization product of said expandable, polymerizable composition as well as a method for manufacturing the polymerization product. The invention also relates to a sealant, adhesive, coating, binding agent or dental filling comprising said expandable, polymerizable composition as well as the use of said expandable, polymerizable composition as/in sealants, adhesives, coatings, binding agents or dental fillings.

Volume shrinkage that occurs during the process of polymerization is often neglected and highly under-represented in the literature. The shrinkage during polymerization originates from the change in the bonding distance from van-der-Waals distances in the monomer (3.4 Å) to covalent distances in the polymer (1.5 Å). Values for polymerization shrinkage can be as high as 50% for polycondensation polymerizations or as low as 2-5% in ring-opening polymerizations.

A great number of currently used polymerizable compositions are predominantly based on epoxides. These epoxy-based compositions exhibit volumetric shrinkage during polymerization resulting in delamination, cracking and formation of voids in the resin. In addition to the shrinking, epoxy-based polymers also have other known disadvantages including a high moisture uptake and limited stability at higher temperatures.

In order to find a solution to this problem, different strategies to reduce the volume shrinkage during polymerizations have been developed. These strategies are mainly based on three approaches: (A) change of polymerization conditions (photopolymerization or thermally cured polymers), (B): addition of fillers or additives, and (C): modification of the structure of the monomer or oligomer.

For example, spiroorthocompounds (SOC compounds) were reported as the monomers with exceptionally high volumetric expansion upon polymerization (T. Takata, T. Endo: Recent advances in the development of expanding monomers: Synthesis, polymerization and volume change. Prog. Polym. Sci. 1993, 18(5), 839-870). However, SOC compounds have the disadvantage of being water sensitive. Another potential issue considering industrial applications is the fact that the synthesis of numerous spiroortho monomers in the literature involves various compounds that are prohibited by REACH (namely the registration, evaluation, authorization and restriction of chemicals).

Another set of compounds that was reported to lead to volume expansion upon curing are benzoxazine-based phenolic resins (H. Ishida, H. Y. Low: A Study on the Volumetric Expansion of Benzoxazine-Based Phenolic Resin. Macromolecules 1997, 30, 1099-1106). Various benzoxazine monomers can be prepared from bisphenolic units (most frequently Bisphenol A), formaldehyde, and primary amines. However, a huge difference among different amine-based benzoxazines in their volumetric expansion characteristics was observed. Even if poly(benzoxazine)s can overcome many of the disadvantages reported for epoxy-based polymers, the methods for producing poly(benzoxazine)s require high temperatures and the resulting polymers are typically brittle, thus, limiting their spectrum of applications.

The volume expansion during the polymerization of cyclic carbonates containing norbornene units (NBC) with an amine-based initiator was reported by Murayama (M. Murayama: Anionic Ring-Opening Polymerization of a Cyclic Carbonate Having a Norbornene Structure with Amine Initiators. Macromolecules 1998, 31, 919-923). The polymerization of pure NBC has been reported to lead to an expansion of 8.2%.

Cho and coworkers studied the effect on the shrinkage of incorporating various lactams into epoxy resins during copolymerization (L. W. Chen, C. H. Twu, C. S. Cho: Physical properties and shrinkage of epoxy resins cured with lactams. J. Polym. Res. 1997, 4, 65-72). The shrinkage decreased with increasing lactam content and with increasing lactam ring size.

Furthermore, it was reported that polyurethane acrylates with different amounts of stearyl alcohols effect the volume shrinkage (L. Qin, J. Nie, Y. He: Synthesis and properties of polyurethane acrylate modified by different contents of stearyl alcohol. J. Coat. Technol. Res. 2015, 12, 197-204). Specifically, polyurethane acrylates modified by a higher stearyl alcohol content exhibit lower volume shrinkage.

As the polymerization shrinkage is directly related to the conversion of reactive double bonds, novel photopolymerizable formulations based on dimethylacrylate monomers with bulky substituent groups were shown to exhibit lower shrinkage upon polymerization (J. Ge, M. Trujillo, J. Stansbury: Synthesis and photopolymerization of low shrinkage methacrylate monomers containing bulky substituent groups. Dent. Mater. 2005, 21, 1163-1169).

Nie and coworkers studied the effect of temperature on the volume shrinkage during the photopolymerization of tri(ethylene glycol) dimethacrylate (B. Lu, P. Xiao, M. Sun, J. Nie: Reducing volume shrinkage by low-temperature photopolymerization. J. Appl. Polym. Sci. 2007, 104, 1126-1130). They found that the optimum temperature for the photocuring is −40° C. as the total conversion after the dark reaction is similar to the one observed at room temperature, while beneficially the shrinkage was significantly lower.

Another study demonstrated that the shrinkage during photopolymerization of (meth)acrylate coatings depends on double bond conversion and the concentration of double bonds: the monomer was mainly responsible for shrinkage due to the inherent high concentration of double bonds (Y. Jian, Y. He, T. Jiang, C. Li, W. Yang, J. Nie: Volume shrinkage of UV-curable coating formulation investigated by real-time laser reflection method. J. Coat. Technol. Res. 2013, 10, 231-237).

Another approach relates to the use of prepolymers to prevent volume shrinkage. The synthesis of novel types of hyperbranched polyesters with a flexible ethoxylated Bisphenol A structure and terminal hydroxyl groups was reported by Serra and coworkers (T. Li, H. Qin, Y. Liu, Y. Zhong, Y. Yu, A. Serra: Hyperbranched polyester as additives in filled and unfilled epoxy-novolac systems. Polymer 2012, 53(25), 5864-5872).

The effect of the TiO₂ filler on the volume shrinkage of epoxy resin composites was studied by Thomas and coworkers (J. Parameswaranpillai, A. George, J. Pionteck, S. Thomas: Investigation of Cure Reaction, Rheology, Volume Shrinkage and Thermomechanical Properties of Nano-TiO₂Filled Epoxy/DDS Composites. J. Polym. 2013, 183463:1-183463:17).

There is a need for alternative or improved expandable, polymerizable compositions that result in polymers having an improved and tunable expansion behavior during polymerization.

It is an object of the present invention to provide a novel expandable, polymerizable composition based on benzoxazines showing improved expansion properties, in particular, showing high, adjustable and reproducible expansion rates during polymerization. It is another object of the present invention to provide a novel expandable, polymerizable composition based on benzoxazines, which results in polymers having a wide spectrum of applications.

These objects are achieved by a novel expandable, polymerizable composition comprising at least one benzoxazine and at least one cyclic carbonate.

The present invention is based on the surprising finding that copolymerizing benzoxazine monomers with cyclic carbonate monomers results in novel copolymers having unforeseeably high expansion rates, wherein the properties (e.g. solid/brittle, solid/soft, rubbery) of the resulting copolymers can be easily and reproducibly tuned/adjusted, depending on the ratio of the benzoxazine equivalents/cyclic carbonate equivalents present in the composition and copolymer, respectively. Thus, the novel copolymers provide significant benefits over the expandable polymers known in the art. Furthermore, the novel expandable, polymerizable compositions and poly(benzoxazine)-co-poly(cyclic carbonate)-polymers obtained therewith have a great potential for offering a wide spectrum of applications. As will be described in more detail below, the copolymer can be prepared by the copolymerization of benzoxazines and cyclic carbonates at a temperature sufficient to initiate copolymerization, wherein both types of monomers undergo ring-opening polymerization.

These surprising findings are evidenced by the experimental data given in the examples below. The results presented in the experimental data show that for most of the copolymers that were tested expansion rates of more than 25% or of even more than 30% were observed. To the inventors' best knowledge, such high expansion rates have never been reported so far for expandable polymers, thus, exceeding the expansion rates of known expandable polymers by more than 300%.

In one aspect of the present invention, the at least one benzoxazine comprised in the expandable, polymerizable composition is a compound of general Formula (I):

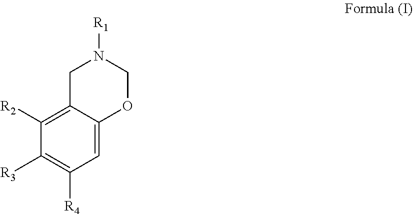

wherein R₁, R₂, R₃, and R₄ are each independently H, CH₃, C₂-C₁₅ straight, branched or cyclic alkyl optionally substituted with halogens, alkenyl or alkynyl groups, alkaryl groups, heteroalkyl groups, heteroaryl groups, hydroxy groups, disulfides, sulfonates, ether groups, thiolether groups, ester groups, carboxylic acid groups, amine groups, amide groups, azides, or benzoxazines, and R₂ can be linked with R₃ to form a cyclic substituent on the benzene ring or R₃ can be linked with R₄ to form a cyclic substituent on the benzene ring.

The term “alkyl” as used herein refers to straight or branched hydrocarbon chains having a specified number of carbon atoms. For example, “C₂-C₁₅ straight, branched or cyclic alkyl” as recited in the claims refers to a straight, branched or cyclic alkyl group having at least 2 and at most 15 carbon atoms. Alkyl groups having a straight chain preferably represent C₂-C₉ alkyl. Useful branched alkyl chains, which preferably represent C₃-C₇ alkyl, are for example substituents of the composition isopropyl, isobutyl, neopentyl, and (1′-methyl)-hexyl. Useful cyclic alkyl chains, which preferably represent C₄-C₁₂ alkyl, are for example substituents of the composition cyclobutyl, cyclohexyl, and adamantyl.

As mentioned above, the C₂-C₁₅ straight, branched or cyclic alkyl is optionally substituted by one or more substituents independently selected from the group consisting of halogens, alkenyl or alkynyl groups, alkaryl groups, heteroalkyl groups, heteroaryl groups, hydroxy groups, disulfides, sulfonates, ether groups, thiolether groups, ester groups, carboxylic acid groups, amine groups, amide groups, azides, or benzoxazines.

The term “halogen” as used herein means the halogens chlorine, bromine, fluorine or iodine, wherein chlorine and bromine are preferred, as well as the pseudo halogens CN, N₃, OCN, NCO, CNO, SCN, NCS, SeCN , wherein N₃, NCO, and SCN, are preferred.

The term “alkenyl” as used herein refers to alkyl chains containing the specified number of carbon atoms and containing at least one carbon-carbon double bond, wherein alkyl is defined as above and may be a straight or a branched hydrocarbon chain or may bear ring closures. For example, C₂-C₂₅ alkenyl means an alkenyl containing at least 2, and at most 25, carbon atoms and containing at least one double bond. Straight alkenyl chains are preferred. Examples of “alkenyl” as used herein include but are not limited to 2-propenyl, Z- or E-propenyl, butadienyl, isoprenyl and cyclopropenylethyl as well as Z- or E-decenyl and E-octadec-9-enyl.

The term “alkinyl” as used herein refers to alkyl chains containing the specified number of carbon atoms and containing at least one carbon-carbon triple bond, wherein alkyl is defined as above and may be a straight or a branched hydrocarbon chain or may bear ring closures. For example, C₂-C₁₂ alkinyl means an alkinyl containing at least 2, and at most 12, carbon atoms and containing at least one triple bond. Representative examples of alkinyl include, but are not limited, to 1-propinyl, 2-propinyl, 3-butinyl and 2-pentinyl as well as 1-dodecinyl.

The term “alkaryl” as used herein refers to a group of the formula alkylene-aryl or arylene-alkyl. In particular, alkaryl relates to an aryl group attached to the benzoxazine ring via an alkylene group. The term “aryl” as used herein refers to an aromatic carbocyclic ring having a specified number of carbon atoms. For example, C₅-C₆ aryl refers to an aromatic ring having at least 5 and at most 6 carbon atoms. The term “aryl” also refers to a multicyclic group having more than one aromatic ring, such as C₁₀-C₁₄ aryl. Representative examples of aryl include, but are not limited to cyclopentadienyl, phenyl, naphthyl, anthracyl, phenanthryl. Exemplary alkaryl groups are from 7 to 16 carbon atoms, and include, but are not limited to benzyl, phenethyl, and isopropylbenzyl.

The term “heteroalkyl” as used herein refers to an “alkyl” group in which at least one carbon atom has been replaced with a heteroatom (e.g., an O, N, or S atom). The heteroalkyl may be, for example, primary, secondary, and tertiary amines, linear, branched, and cyclic thioethers, primary thiols, linear, branched, and cyclic ethers as well as carboxylic acids and esters derived thereof. In certain embodiments, the “heteroalkyl” may be 2-8 membered heteroalkyl, indicating that the heteroalkyl contains from 2 to 8 atoms selected from the group consisting of carbon, oxygen, nitrogen, and sulfur. In yet other embodiments, the heteroalkyl may be a 2-6 membered, 4-8 membered, or a 5-8 membered heteroalkyl group (which may contain for example 1 or 2 heteroatoms selected from the group oxygen and nitrogen).

The term “heteroaryl” as used herein refers to aromatic groups having one or more hetero-atoms (O, S or N). Preferred heteroaryl residues have 5 or 6 ring members and include those derived from furan, imidazole, triazole, isothiazole, isoxazole, oxadiazole, oxazole, pyrazine, pyrazole, pyridazine, pyridine, pyrimidine, pyrroline, thiazole, thiophene, triazole, and tetrazole. A multicyclic group having one or more heteroatoms, wherein at least one ring of the group is heteroaromatic, is referred to as a “heteroaryl” as well; for example heteroaryl groups having 7 to 10 ring members and including those derived from indole, quinoline, isoquinoline and tetrahydroquinoline.

The terms “hydroxy”, “disulfides”, “sulfonates”, “thioether” “ether”, “ester” and “carboxylic acid” are art recognized. Exemplary disulfides include, but are not limited to methyl disulfanyl, ethyl disulfanyl, phenyl disulfanyl and benzyl disulfanyl. Exemplary sulfonates include, but are not limited to mesylate, tosylate, sodium methanesulfonate, potassium methanesulfonate, and sodium 2-methyl benzenesulfonate. Exemplary ether groups include, but are not limited to methoxy, ethoxy, benzoxy, and phenoxy. Exemplary thioether groups include, but are not limited to methylthiyl, ethylthiyl, and phenylthiyl. Exemplary ester groups include, but are not limited to formiates, acetates, propanoates, phthalates, and benzoates. Exemplary carboxylic acid groups include, but are not limited to formic acids, acetic acids, benzoic acids, phthalic acids, and fatty acids such as decanoic acids and dec-10′-enoic acid.

The terms “amine” and “amino” are art-recognized and refer to primary, secondary and tertiary amines, including, but not limited to ethylamine, benzylamine, ethylenediamine, isophoron diamine, diethylene triamine, tert-butyl amine, and triethylamine.

The term “amide” as used herein, represents a molecule having at least one identifiable amide group. Exemplary amides include, but are not limited to acetamidomethyl, propionamidomethyl, and benzamidomethyl.

The term “azide” as used herein refers to any group having the N₃ moiety therein. Exemplary azides include, but are not limited to (4H-1,2,3-triazol-4-yl)methyl, (5-methyl-4H-1,2,3-triazol-4-yl)methyl, (5-ethyl-4H-1,2,3-triazol-4-yl)methyl, and (5-phenyl-4H-1,2,3-triazol-4-yl)methyl, as well as azidomethyl, azidoethyl, azidopropyl, 4-azidophenyl, and 4-azidobenzyl.

As used herein, the term “benzoxazine” relates to compounds and polymers comprising the characteristic benzoxazine ring. Exemplary benzoxazine substituents include, but are not limited to (2H-benzo [e][1,3]oxazin-3(4H)-yl)methyl, (5,6,7-trimethyl-2H-benzo [e][1,3]oxazin-3(4H)-yl)methyl, (7-amino-5-azido-6-hydroxy-2H-benzo [e][1,3]oxazin-3(4H)-yl)methyl, and 2-((2H-benzo[e][1,3]oxazin-3(4H)-yl)methyl)benzyl. For example, the synthesis of benzoxazine monomers of Formula (I) wherein one of R₁, R₂, R₃, and R₄ is C₂-C₁₅ straight, branched or cyclic alkyl substituted with a benzoxazine, may be based on a symmetric bisphenol compound or a dihydroxybenzene compound.

The term “substituted” refers to a chemical entity being substituted with one or more substituents. The term “optionally substituted” relating to a chemical entity means that the chemical entity may be substituted or not, i.e. both situations are included. The term “one or more substituents” refers to from one to the maximum number of substituents possible, depending on the number of available substitution sites.

In certain embodiments, R₂ can be linked with R₃ to form a cyclic substituent on the benzene ring. In other embodiments, R₃ can be linked with R₄ to form a cyclic substituent on the benzene ring. Exemplary cyclic substituents include, but are not limited to 3-allyl-1,3-oxazinane-5,6-diyl, 3-allyl-4-methyl-1,3-oxazinane-5,6-diyl, cyclohexane-1,2-diyl, and benzene-1,2-diyl. For example, by linking R₂ with R₃ or by linking R₃ with R₄ a cyclic substituent in form of a 4-, 5- or 6-membered alkyl-ring may be formed.

In another aspect, the benzoxazine units comprised in the composition are benzoxazine units which are crosslinkable with each other. Preferably the benzoxazines are crosslinkable by thiol-ene crosslinking. In certain embodiments the crosslinkable benzoxazine is a compound of general Formula (II):

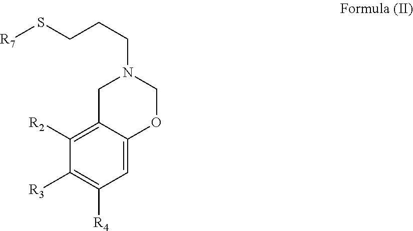

wherein R₂, R₃, and R₄ are each independently H, CH₃, C₂-C₁₅ straight, branched or cyclic alkyl optionally substituted with halogens, alkenyl or alkynyl groups, alkaryl groups, heteroalkyl groups, heteroaryl groups, hydroxy groups, disulfides, sulfonates, ether groups, thioether groups, ester groups, carboxylic acid groups, amine groups, amide groups, azides, or benzoxazines, and R₂ can be linked with R₃ to form a cyclic substituent on the benzene ring or R₃ can be linked with R₄ to form a cyclic substituent on the benzene ring, with the proviso that R₂, R₃, and/or R₄ comprises at least one unsaturated bond, e.g. an olefinic bond, and

R₇ is C₂-C₁₅ straight, branched or cyclic alkyl optionally substituted with halogens, alkaryl groups, heteroalkyl groups, heteroaryl groups, hydroxy groups, disulfides, sulfonates, ether groups, thioether groups, ester groups, carboxylic acid groups, amine groups, amide groups and azides, with the proviso that R₇ comprises at least one thiol group in order to enable (UV-mediated) thiol-ene crosslinking.

UV-mediated crosslinking of monomers comprising available alkene and thiol functional groups via thiol-ene reactions (also known as “thiol-ene click chemistry”) is well known to those skilled in the art and frequently used in the synthesis of crosslinked three-dimensional networks. In the present invention, the benzoxazine units may be crosslinked by a UV-initiated thiol-ene click reaction prior to the copolymerization. The copolymerization of the crosslinked benzoxazine units and the cyclic carbonate units, where expansion of the resulting copolymer takes place, is performed by applying heat at a temperature sufficient to initiate copolymerization, which temperature is e.g. a temperature of up to 140° C. in case of the present invention.

The at least one cyclic carbonate may be a compound of general Formula (III):

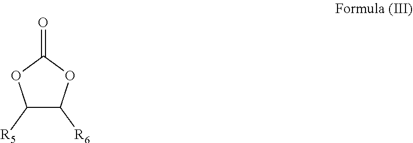

wherein R₅ and R₆ are each independently H, CH₃, C₂-C₁₅ straight, branched or cyclic alkyl optionally substituted with halogens, alkenyl, heteroalkyl groups and hydroxy groups. In preferred embodiments, the cyclic carbonate is ethylene carbonate or propylene carbonate, preferably ethylene carbonate.

In specific embodiments, the benzoxazine may be derived from a dihydroxybenzene or bisphenol. Preferably the dihydroxybenzene is hydroquinone, and the bisphenol is selected from the group consisting of Bisphenol A, Bisphenol F, Bisphenol S, Bisphenol M, Bisphenol Z, and Bisphenol AP. Examples of such benzoxazines are given in the experimental part in the examples below.

The ratio of benzoxazine equivalents to cyclic carbonate equivalents in the composition is preferably from 99:1 to 1:99; more preferably, the ratio of benzoxazine equivalents to cyclic carbonate equivalents in the composition is from 99:1 to 30:70. Depending on the ratio, the characteristics (e.g. solid/brittle, solid/soft, rubbery) of the polymerization product obtained after polymerizing the composition according to the invention can be adjusted/tuned.

In other embodiments, the composition additionally comprises at least one reactive diluent. In specific embodiments, the reactive diluent is present at between 20% to 60% by weight of the benzoxazine. In specific embodiments, the reactive diluent is selected from the group consisting of 3-allyl-3,4-dihydro-2H-benzo[e][1,3]oxazine, 3-allyl-5-methyl-3,4-dihydro-2H-benzo [e][1,3]oxazine, 3-allyl-6-octyl-3,4-dihydro-2H-benzo [e][1,3]oxazine, and 3-allyl-6-nonyl-3,4-dihydro-2H-benzo[e][1,3]oxazine.

The present invention also relates to a polymerization product of an expandable, polymerizable composition according to the present invention and described herein.

In certain embodiments, the polymerization product according to the invention comprises a poly(benzoxazine)-co-poly(cyclic carbonate) of the general formula (IV):

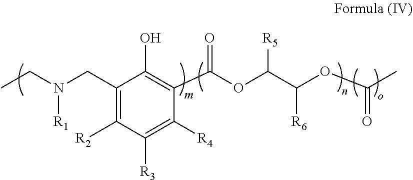

wherein

R₁, R₂, R₃, and R₄ are each independently H, CH₃, C₂-C₁₅ straight, branched or cyclic alkyl optionally substituted with halogens, alkenyl or alkynyl groups, alkaryl groups, heteroalkyl groups, heteroaryl groups, hydroxy groups, disulfides, sulfonates, ether groups, thioether groups, ester groups, carboxylic acid groups, amine groups, amide groups, azides, or benzoxazine, and R₂ can be linked with R₃ to form a cyclic substituent on the benzene ring or R₃ can be linked with R₄ to form a cyclic substituent on the benzene ring;

R₅ and R₆ are each independently H, CH₃, C₂-C₁₅ straight, branched or cyclic alkyl optionally substituted with halogens, alkenyl, heteroalkyl groups and hydroxy groups;
m is an integer between 10 and 10′000;
n is an integer between 10 and 6′700;
o is an integer between 0 and 1′000.

During copolymerization, both types of monomers, i.e. the benzoxazine monomers and cyclic carbonate monomers, undergo ring-opening polymerization as depicted below:

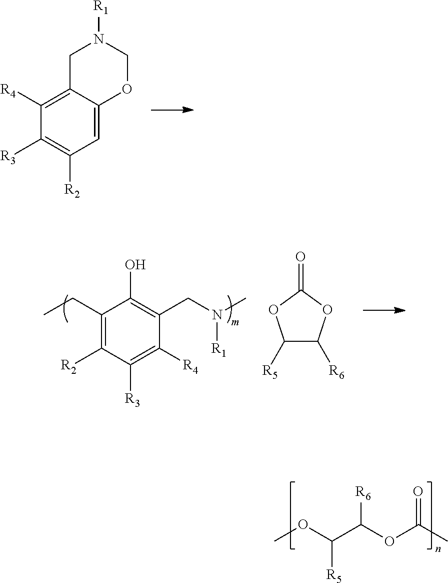

The copolymerization step for obtaining a poly(benzoxazine)-co-poly(cyclic carbonate) of the above-mentioned general formula (IV) is depicted below:

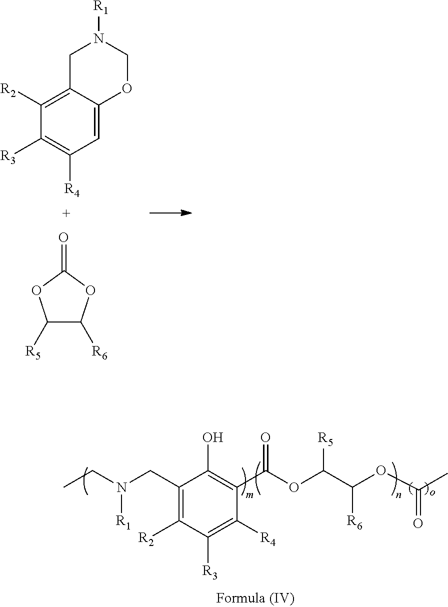

In certain embodiments, the polymerization product of the present invention comprises a poly(benzoxazine)-co-poly(cyclic carbonate) of the general formula (V):

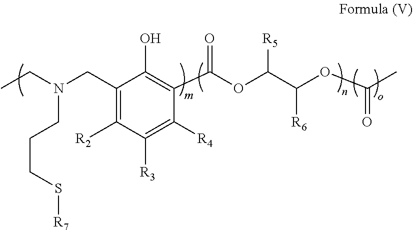

wherein

R₂, R₃, and R₄ are each independently H, CH₃, C₂-C₁₅ straight, branched or cyclic alkyl optionally substituted with halogens, alkenyl or alkynyl groups, alkaryl groups, heteroalkyl groups, heteroaryl groups, hydroxy groups, disulfides, sulfonates, ether groups, thioether groups, ester groups, carboxylic acid groups, amine groups, amide groups, azides, or benzoxazine, and R₂ can be linked with R₃ to form a cyclic substituent on the benzene ring or R₃ can be linked with R₄ to form a cyclic substituent on the benzene ring, with the proviso that R₂, R₃, and/or R₄ comprises at least one unsaturated bond;

R₇ is C₂-C₁₅ straight, branched or cyclic alkyl optionally substituted with halogens, alkaryl groups, heteroalkyl groups, heteroaryl groups, hydroxy groups, disulfides, sulfonates, ether groups, thioether groups, ester groups, carboxylic acid groups, amine groups, amide groups and azides, with the proviso that R₇ comprises at least one thiol group, in order to enable (UV-mediated) thiol-ene crosslinking.

R₅ and R₆ are each independently H, CH₃, C₂-C₁₅ straight, branched or cyclic alkyl optionally substituted with halogens, alkenyl, heteroalkyl groups and hydroxy groups;
m is an integer between 10 and 10′000;
n is an integer between 10 and 6′700;
o is an integer between 0 and 1000.

The two-step polymerization for obtaining a poly(benzoxazine)-co-poly(cyclic carbonate) of the above-mentioned general formula (V) is depicted below:

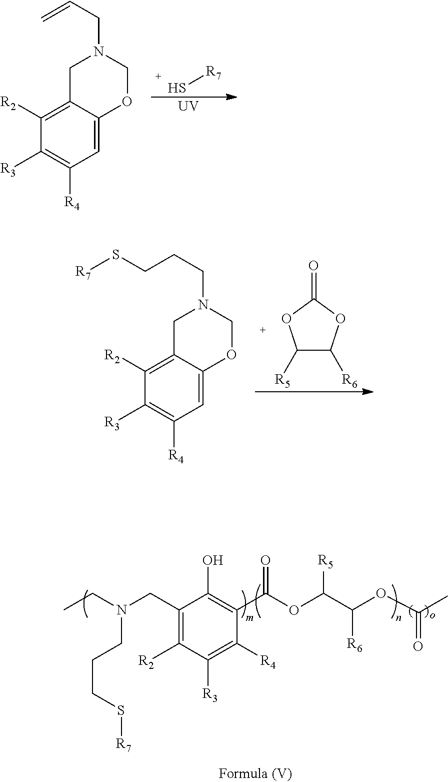

In these embodiments, the benzoxazine units are first be crosslinked by a UV-initiated thiol-ene click reaction prior to copolymerizing the crosslinked benzoxazine units and the cyclic carbonates. The copolymerization, during which expansion of the resulting copolymer takes place, is performed by applying heat at a temperature sufficient to initiate copolymerization, which temperature is e.g. a temperature of up to 140° C. in case of the present invention.

In a further aspect, the present invention also relates to a sealant, an adhesive, a coating, a binding agent or a dental filling comprising an expandable polymerizable composition as described herein. In yet another aspect, the present invention also relates to the use of an expandable, polymerizable composition as described herein as/in sealants, adhesives, coatings, binding agents or dental fillings. For example, the expandable polymerizable composition may be useful in the building industry, e.g. as casting compositions and sealing agents; in the casting industry, e.g. as binders and moulding mixtures; in the coating industry, e.g. for coating of objects; in the wood processing industry, e.g. for the preparation of pressed wood products; in the automotive industry; in the electronic industry, e.g. as insulating materials; in the adhesives industry; and in the field of medical/dental products, e.g. dental fillings.

In a further aspect, the present invention relates to a precured, expandable, polymerizable composition obtained by crosslinking crosslinkable benzoxazines comprised in an expandable polymerizable composition as described herein. In a further aspect, the present invention also relates to the use of this precured, expandable, polymerizable composition, as an expandable filling element for filling spaces in devices, wherein the precured, expandable composition has a pre-defined form, in particular, but not limited to, bars, sticks, cylinders or building blocks, wherein the precured, expandable composition shows volumetric expansion during copolymerization upon a heat stimulus. In specific embodiments, the volumentric expansion of the pre-formed and precured composition may result in a geometric alignment with the space to be filled. Said pre-defined forms of the precured, expandable, polymerizable composition may have dimensions in the milli-, centi-, or meter range. Specifically, said precured, expandable, polymerizable composition may be used as a support and fixation element for windings of electrical machines or may serve as a winding insulation barrier for electrical equipment. That is, due to the volumetric expansion, precise geometric alignment of the resulting expanded copolymer to the surrounding material occurs and compensates the shrinkage and assembling tolerances of those materials, which renders excellent stability and, additionally, insulating properties and form fit support of the windings or laminated electromagnetic materials (see also chapter 4 of the applications examples below).

In yet another aspect, the present invention also relates to a process for manufacturing a polymerization product, comprising the step of heating an expandable, polymerizable composition as described herein to a temperature sufficient to initiate copolymerization. The temperature at which copolymerization is initiated is preferably in the range of 70-200° C., more preferably in the range of 70-150° C., and most preferably in the range of 70-120° C.

In specific embodiments of the process for manufacturing a polymerization product, the process comprises the steps of:
a.) providing a polymerizable composition according to claim 3 or 4, comprising crosslinkable benzoxazines and cyclic carbonates,
b.) precuring the polymerizable composition by crosslinking the crosslinkable benzoxazines, and
c.) heating the precured polymerizable composition to a temperature sufficient to initiate copolymerization of the crosslinked benzoxazines and cyclic carbonates.

Thus, as described above, step b.) precuring of the expandable, polymerizable composition may be performed by UV-mediated crosslinking of the crosslinkable benzoxazine monomers comprising available alkene and thiol functional groups via thiol-ene reactions prior to the copolymerization step c.), in which the expansion of the poly(benzoxazine)-co-poly(cyclic carbonate) takes place. In copolymerization step c.) heat at a temperature sufficient to initiate copolymerization is applied. The temperature at which copolymerization is initiated is preferably in the range of 70-200° C., more preferably in the range of 70-150° C., and most preferably in the range of 70-120° C. This two-step process comprising a precuring step b.) followed by a copolymerization/expansion step c.) is particularly useful in applications in the building and casting industry, since it allows for an easy and convenient handling/arrangement of the precured, thus preformed, but not yet expanded, composition. For example, the precured composition that has already a desired shape may be placed in an opening to be sealed; in a next step, the application of heat initiates copolymerization and expansion of the resulting crosslinked poly(benzoxazine)-co-poly(cyclic carbonate).

The present invention is further demonstrated and illustrated by the following examples, yet without being restricted thereto.

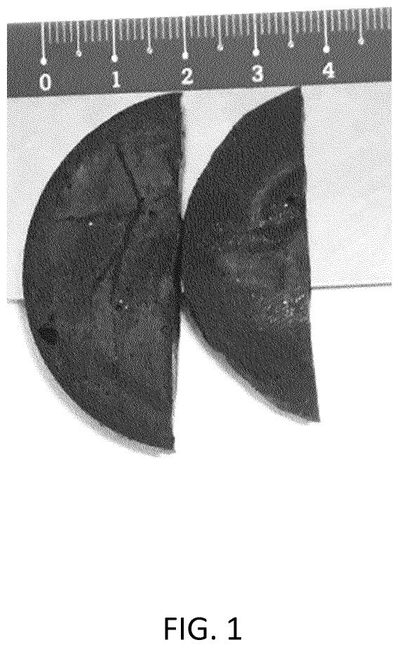

FIG. 1 of chapter 3.1 below illustrates a thiol-ene precured but not copolymerized specimen (on the right) and a thiol-ene precured and copolymerized specimen (on the left), wherein both specimen are prepared with an expandable, polymerizable composition according to the invention.

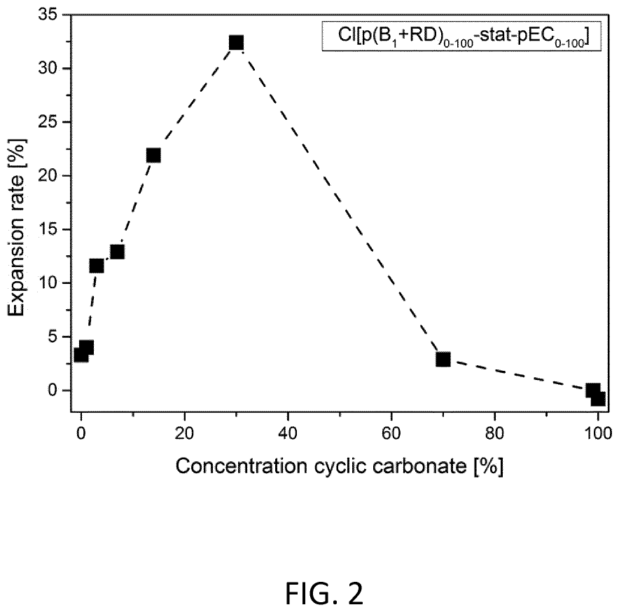

FIG. 2 shows the expansion rate in [%] as a function of the concentration of the cyclic carbonate in the copolymer of Cl[p(B₁+RD)₁₋₁₀₀-stat-pEC₁₋₁₀₀]; see chapter 3.B.1 below.

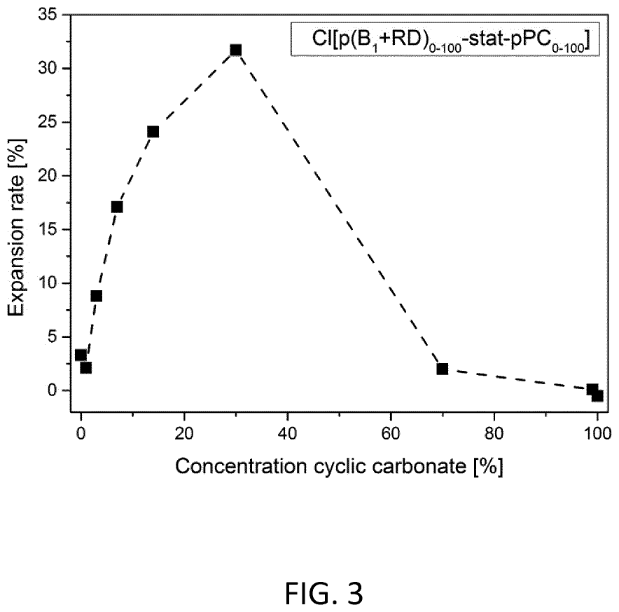

FIG. 3 shows the expansion rate in [%] as a function of the concentration of the cyclic carbonate in the copolymer Cl[p(B₁+RD)₁₋₁₀₀-stat-pPC₀₋₁₀₀]; see chapter 3.B.2. below.

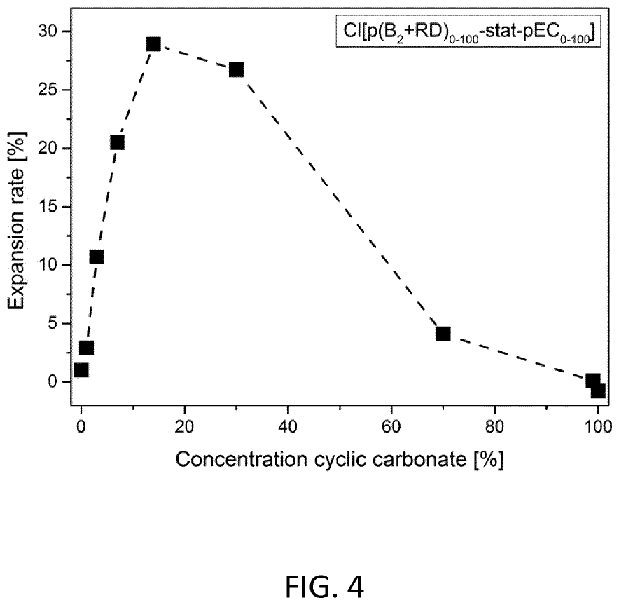

FIG. 4 shows the expansion rate in [%] as a function of the concentration of the cyclic carbonate in the copolymer Cl[p(B₂+RD)₀₋₁₀₀-stat-pEC₀₋₁₀₀]; see chapter 3.B.3. below.

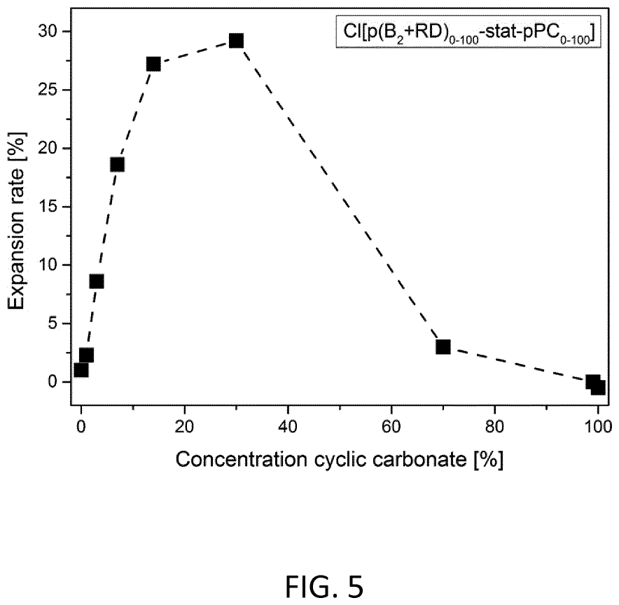

FIG. 5 shows the expansion rate in [%] as a function of the concentration of the cyclic carbonate in the copolymer Cl[p(B₂+RD)₀₋₁₀₀-stat-pPC₀₋₁₀₀]; see chapter 3.B.4. below.

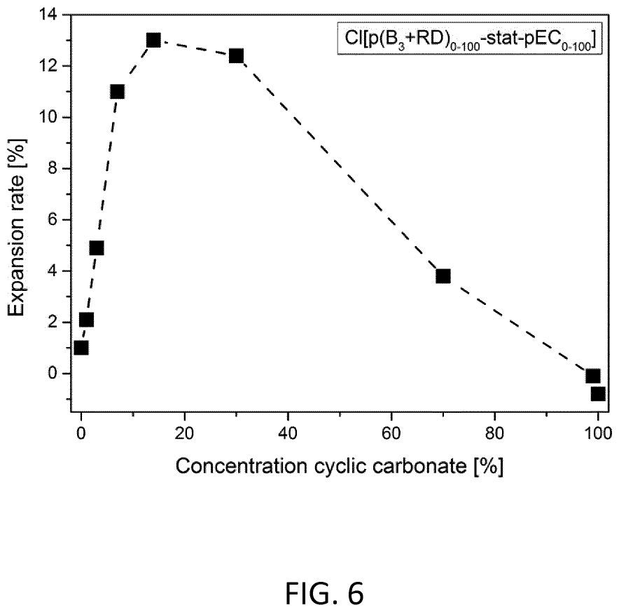

FIG. 6 shows the expansion rate in [%] as a function of the concentration of the cyclic carbonate in the copolymer Cl[p(B₃+RD)₀₋₁₀₀-stat-pEC₁₋₁₀₀]; see chapter 3.B.5. below.

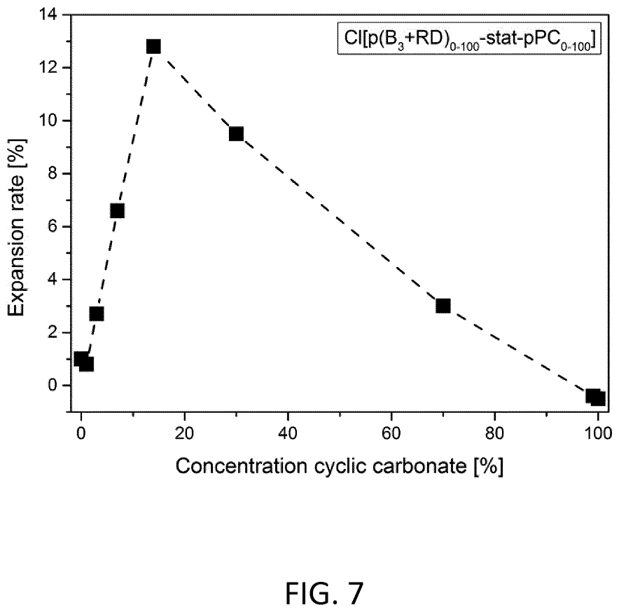

FIG. 7 shows the expansion rate in [%] as a function of the concentration of the cyclic carbonate in the copolymer Cl[p(B₃+RD)₀₋₁₀₀-stat-pPC₀₋₁₀₀]; see chapter 3.B.6. below.

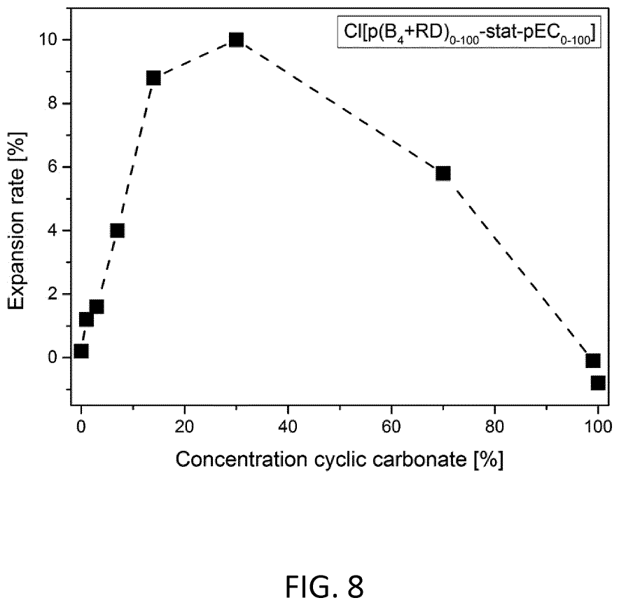

FIG. 8 shows the expansion rate in [%] as a function of the concentration of the cyclic carbonate in the copolymer Cl[p(B₄+RD)₀₋₁₀₀-stat-pEC₀₋₁₀₀]; see chapter 3.B.7. below.

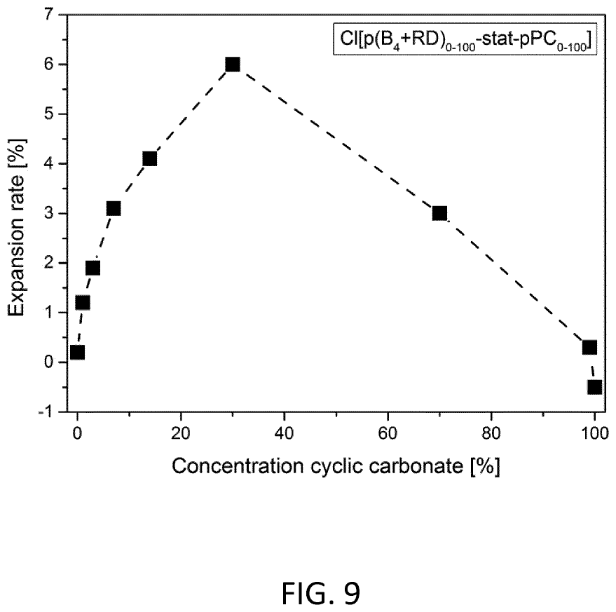

FIG. 9 shows the expansion rate in [%] as a function of the concentration of the cyclic carbonate in the copolymer Cl[p(B₄+RD)₀₋₁₀₀-stat-pPC₀₋₁₀₀]; see chapter 3.B.8. below.

### EXPERIMENTAL DATA and EXAMPLES

### 1. A. Materials and Methods

### 1.A.1 Materials

Bisphenol A (97%), hydroquinone (97%), para-formaldehyde (95%), phenol (>99%), m-cresol (>98%), 4-octylphenol (99%), 4-nonylphenol (>90%), sodium sulfate (>99%), ethylene carbonate (98%), propylene carbonate (99.7%), and diethyl ether (>98%) were purchased from Sigma-Aldrich Chemie GmbH (Vienna, Austria). Bisphenol F (>99%), Bisphenol S (>98%), pentaerythritol tetrakis(3-mercaptopropionate) (>90%) were purchased from TCI (Tokyo Chemical Industry Co., Ltd.; Austria, Vienna). Allylamine (>98%) was purchased from Thermo Fisher GmbH (Karlsruhe, Germany). Ethyl 2,4,6-trimethylbenzoylphenylphosphinate (>95%) was purchased from abcr GmbH (Karlsruhe, Germany). All substances were used without further purification.

### 1.A.2 Methods

### Measurement of Expansion Rates—Density Measurements

Density measurements were performed using a Mettler Toledo density measurement kit. The sample density is calculated with a hydrostatic balance in water, determining the uplift of specimens in and out of the solvent.

Expansion rates were quantified by density measurements of the monomers/precursors on the one hand and the expanded polymers on the other. Expansion rates were calculated according to the following formula:

$$Δ V ( \% ) = \frac{δ ( Mono . ) - δ ( Polym . )}{δ ( Polym . )} \times 100$$

FTIR, ¹H-NMR and ¹³C-NMR Measurements

FTIR-ATR spectra were recorded on a Bruker Alpha P instrument equipped with a DTGS detector (spectral range between 4000 and 800 cm⁻¹). The ATR unit is equipped with a diamond crystal. FTIR spectra were measured in transmission mode and obtained from powdered samples or liquid films.

NMR spectra were recorded on a Bruker Ultrashield 300 WB 300 MHz spectrometer. The solvent peak of CDCl₃ served as reference of the spectra (7.26 ppm for ¹H and 77.0 ppm for ¹³C). The solvent residual peak of DMSO was used for referencing the spectra to 2.50 (¹H) and 39.5 (¹³C) ppm. Peak shapes are indicated as follows: s (singlet), d (doublet), t (triplet), m (multiplet).

### 2.A. Monomer Synthesis

2.A.1 6,6′-(propane-2,2-diyl)bis(3-allyl-3,4-dihydro-2H-benzo[e][1,3]oxazine)

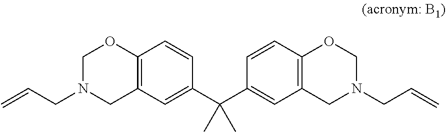 [SMILES: C=CCN1COc2ccc(C(C)(C)c3ccc4c(c3)CN(CC=C)CO4)cc2C1]

To a mixture of Bisphenol A (0.15 mol, 34.3 g) and allylamine (0.30 mol, 17.1 g), para-formaldehyde (0.62 mol, 18.6 g) is added in small portions over a period of 30 min, while being cooled in an ice bath to keep the temperature below 10° C. Then, para-toluene sulfonic acid (2.90 mmol, 0.50 g) is added, the temperature is raised to 90° C., and the mixture is stirred for 3 h. The resulting reaction mixture is dissolved in 500 mL of diethyl ether. The ether solution is washed with water (3 portions of 500 mL) to eliminate any unreacted formaldehyde, Bisphenol A or allyl amine and dried over sodium sulfate. The solvent is evaporated under reduced pressure and the product dried in a vacuum oven.

¹H-NMR (300 MHz, CDCl₃): δ (ppm)=6.86 (2 H, m), 6.72 (2 H, m), 6.62 (2 H, m), 5.80 (2 H, m), 5.12 (4 H, m), 4.74 (4 H, s), 3.85 (4 H, s), 3.31 (2 H, d), 1.51 (6 H, s).

¹³C-NMR (75 MHz, CDCl₃): δ (ppm)=151.9, 143.8, 135.1, 126.3, 122.5, 119.2, 118.3, 115.8, 81.9, 54.5, 50.3, 41.7, 31.1.

FTIR (ATR): v (cm⁻¹)=3074, 2964, 2894, 2825, 1611, 1495, 1330, 1226, 1187, 1115, 987, 928, 814.
2.A.2 Bis(3-allyl-3,4-dihydro-2H-benzo[e][1,3]oxazin-6-yl)methane

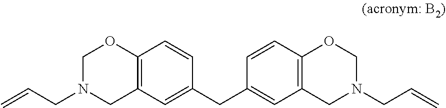 [SMILES: C=CCN1COc2ccc(Cc3ccc4c(c3)CN(CC=C)CO4)cc2C1]

To a mixture of Bisphenol F (0.15 mol, 29.7 g) and allylamine (0.30 mol, 17.1 g), para-formaldehyde (0.62 mol, 18.6 g) is added in small portions over a period of 30 min, while being cooled in an ice bath to keep the temperature below 10° C. Then, para-toluene sulfonic acid (2.90 mmol, 0.50 g) is added, the temperature is raised to 90° C., and the mixture is stirred for 3 h. The resulting reaction mixture is then dissolved in 500 mL of diethyl ether. The ether solution is washed with water (3 portions of 500 mL) to eliminate any unreacted para-formaldehyde, Bisphenol F or allyl amine, and dried over sodium sulfate. The solvent is evaporated under reduced pressure and the product is dried in a vacuum oven.

¹H-NMR (300 MHz, CDCl₃): δ (ppm)=6.83 (2 H, m), 6.67 (2 H, m), 6.60 (2 H, m), 5.79 (2 H, m), 5.10 (4 H, m), 4.73 (4 H, s), 3.84 (4 H, s), 3.66 (2 H, s), 3.30 (2 H, d).

¹³C-NMR (75 MHz, CDCl₃): δ (ppm)=152.5, 135.3, 133.7, 128.2, 127.7, 120.1, 118.4, 116.5, 82.5, 55.6, 49.8, 40.5.

FTIR (ATR): v (cm⁻¹)=3074, 2972, 2917, 2863, 1740, 1619, 1493, 1436, 1368, 1211, 1109, 987, 928, 811.

2.A.3 6,6′-sulfonylbis(3-allyl-3,4-dihydro-2H-benzo[e][1,3]oxazine)

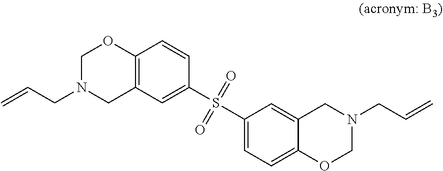 [SMILES: C=CCN1COc2ccc(S(=O)(=O)c3ccc4c(c3)CN(CC=C)CO4)cc2C1]

To a mixture of para-formaldehyde (0.62 mol, 18.6 g) and allylamine (0.30 mol, 17.1 g), Bisphenol S (0.15 mol, 39.3 g) is added in small portions over a period of 30 min, while being stirred at 60° C. Then, para-toluene sulfonic acid (2.90 mmol, 0.50 g) is added, the temperature is raised to 90° C., and the mixture stirred for 3 h. The resulting reaction mixture is then dissolved in 500 mL of chloroform. The solution is washed with water (3 portions of 500 mL) to eliminate any unreacted para-formaldehyde, Bisphenol S or allyl amine and dried over sodium sulfate. The solvent is evaporated under reduced pressure, and the product is dried in a vacuum oven.

¹H-NMR (300 MHz, CDCl₃): δ (ppm)=7.83 (2 H, m), 7.68 (2 H, m), 5,90 (2 H, m), 5.21 (4 H, m), 4.81 (4 H, s), 4.07 (4 H, s), 3.32 (4 H, d).

¹³C-NMR (75 MHz, DMSO): δ (ppm)=163.4, 135.4, 134.9, 130.4, 128.2, 122.2, 118.5, 117.2, 55.1, 49.8.

FTIR (ATR): v (cm⁻¹)=3072, 2958, 2007, 2850, 1683, 1577, 1484, 1440, 1287, 1442, 1081, 991, 916, 822.

2.A.4 2,9-diallyl-1,2,3,8,9,10-hexahydrobenzo[2,1-e:3,4-e′]bis([1,3]oxazine)

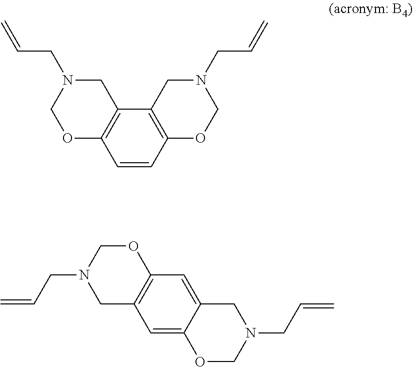

To a mixture of hydroquinone (0.15 mol, 16.5 g) and allylamine (0.30 mol, 17.1 g), para-formaldehyde (0.62 mol, 18.6 g) is added in small portions over a period of 30 min, while being cooled in an ice bath to keep the temperature below 10° C. Then, para-toluene sulfonic acid (2.90 mmol, 0.50 g) is added, the temperature is raised to 90° C., and the mixture is stirred for 3 h. The resulting brown reaction mixture is dissolved in 500 mL of diethyl ether. The ether solution is washed with water (3 portions of 500 mL) to eliminate any unreacted para-formaldehyde, hydroquinone or allyl amine and dried over sodium sulfate. The solvent is evaporated under reduced pressure and the product is dried in a vacuum oven.

¹H-NMR (300 MHz, CDCl₃): δ (ppm)=6.61 (2 H, m), 6.43 (2 H, m), 6.92 (2 H, d), 5.87 (2 H, m), 5.18 (4 H, m), 4.92 (4 H, s), 4.75 (4 H, s), 3.78 (4 H, s), 3.68 (4 H, s), 3.36 (4 H, d), 3.14 (4 H, d).

¹³C-NMR (75 MHz, DMSO): δ (ppm)=150.3, 147.7, 135.0, 118.4, 117.9, 117.5, 117.0, 116.4, 115.6, 115.0, 114.4, 113.0, 81.6, 67.1, 55.8, 54.5, 49.7, 46.6.

FTIR (ATR): v (cm⁻¹)=3076, 2970, 2850, 1740, 1475, 1366, 1230, 1117, 985, 916, 805.

2.A.5 3-allyl-3,4-dihydro-2H-benzo[e][1,3]oxazine (reactive diluent)

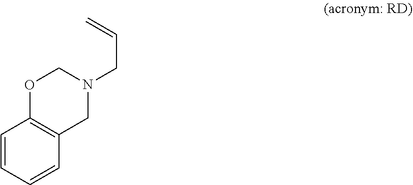

To a mixture of phenol (0.30 mol, 28.2 g) and allylamine (0.30 mol, 17.1 g), para-formaldehyde (0.62 mol, 18.6 g) is added in small portions over a period of 30 min, while being cooled in an ice bath to keep the temperature below 10° C. Then, para-toluene sulfonic acid (2.90 mmol, 0.50 g) is added, the temperature is raised to 90° C., and the mixture is stirred for 3 h. The resulting brown reaction mixture is then dissolved in 500 mL of diethyl ether. The ether solution is washed with water (3 portions of 500 mL) to eliminate any unreacted para-formaldehyde, phenol or allyl amine and dried over sodium sulfate. The solvent is evaporated under reduced pressure and the product is dried in a vacuum oven.

¹H-NMR (300 MHz, CDCl₃): δ (ppm)=7.02 (1 H, m), 6.87 (1 H, m), 6.78 (1 H, m), 6.69 (1 H, m), 5.81 (1 H, m), 5.14 (2 H, m), 4.79 (2 H, s), 3.91 (2 H, s), 3.30 (2 H, d).

¹³C-NMR (75 MHz, CDCl₃): δ (ppm)=158.1, 134.4, 130.1, 129.7, 127.6, 121.0, 117.2, 111.9, 85.4, 58.5, 56.9.

FTIR (ATR): v (cm⁻¹)=3072, 2970, 2841, 1740, 1576, 1487, 1364, 1219, 1107, 991, 922, 850, 754.

2.A.6 3-allyl-5-methyl-3,4-dihydro-2H-benzo[e][1,3]oxazine (reactive diluent)

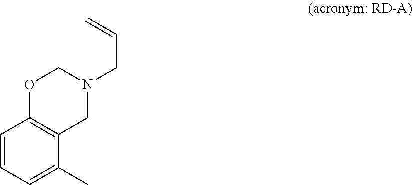

To a mixture of m-cresol (0.30 mol, 32.4 g) and allylamine (0.30 mol, 17.1 g), para-formaldehyde (0.62 mol, 18.6 g) is added in small portions over a period of 30 min, while being cooled in an ice bath to keep the temperature below 10° C. Then, para-toluene sulfonic acid (2.90 mmol, 0.50 g) is added, the temperature is raised to 90° C., and the mixture is stirred for 3 h. The resulting brown reaction mixture is then dissolved in 500 mL of diethyl ether. The ether solution is washed with water (3 portions of 500 mL) to eliminate any unreacted formaldehyde, m-cresol or allyl amine and dried over sodium sulfate. The solvent is evaporated under reduced pressure and the product is dried in a vacuum oven.

¹H-NMR (300 MHz, CDCl₃): δ (ppm)=6.72 (1 H, m), 6.64 (1 H, m), 6.53 (1 H, m), 5.82 (1 H, m), 5.10 (2 H, m), 4.81 (2 H, s), 3.86 (2 H, s), 3.08 (2 H, d), 2.17 (3 H, s).

¹³C-NMR (75 MHz, CDCl₃): δ (ppm)=157.9, 133.4, 130.2, 127.4, 122.2, 121.5, 118.3, 114.2, 82.1, 56.3, 49.4, 21.2.

FTIR (ATR): v (cm⁻¹)=2956, 2843, 1615, 1578, 1505, 1445, 1420, 1279, 1241, 1109, 990, 921, 860.

2.A.7 3-allyl-6-octyl-3,4-dihydro-2H-benzo[e][1,3]oxazine (reactive diluent)

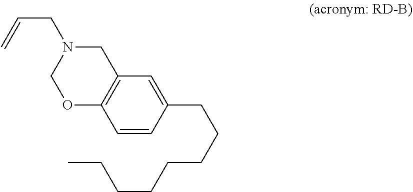 [SMILES: C=CCN1COc2ccc(CCCCCCCC)cc2C1]

To a mixture of 4-octylphenol (0.30 mol, 61.8 g) and allylamine (0.30 mol, 17.1 g), para-formaldehyde (0.62 mol, 18.6 g) is added in small portions over a period of 30 min, while being cooled in an ice bath to keep the temperature below 10° C. Then, para-toluene sulfonic acid (2.90 mmol, 0.50 g) is added, the temperature is raised to 90° C., and the mixture is stirred for 3 h. The resulting brown reaction mixture is then dissolved in 500 mL of diethyl ether. The ether solution is washed with water (3 portions of 500 mL) to eliminate any unreacted para-formaldehyde, 4-octylphenol or allyl amine and dried over sodium sulfate. The solvent is evaporated under reduced pressure and the product is dried in a vacuum oven.

¹H-NMR (300 MHz, CDCl₃): δ (ppm)=6.81 (1 H, d, ³J_{H,H}=6.65 Hz), 6.64 (1 H, s), 6.60 (1 H, ³J_{H,H}=6.65 Hz), 5.85 (1 H, m), 5.12 (2 H, m), 4.84 (2 H, s), 3.88 (2 H, s), 3.21 (2 H, d), 2.41 (2 H, t), 1.48 (2 H, m), 1.19 (10 H, m), 0.79 (2 H, m).

¹³C-NMR (75 MHz, CDCl₃): δ (ppm)=152.0, 135.3, 135.1, 127.6, 127.2, 118.1, 116.2, 82.0, 54.6, 49.7, 35.3, 31.9, 31.7, 29.5, 29.4, 29.3, 22.7, 14.1.

FTIR (ATR): v (cm⁻¹)=2923, 2852, 1499, 1217, 1117, 988, 919, 820.

2.A.8 3-allyl-6-nonyl-3,4-dihydro-2H-benzo[e][1,3]oxazine (reactive diluent)

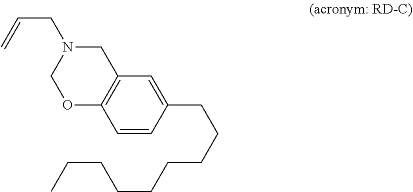 [SMILES: C=CCN1COc2ccc(CCCCCCCCC)cc2C1]

To a mixture of 4-nonylphenol (0.30 mol, 66.1 g) and allylamine (0.30 mol, 17.1 g), para-formaldehyde (0.62 mol, 18.6 g) is added in small portions over a period of 30 min, while being cooled in an ice bath to keep the temperature below 10° C. Then, para-toluene sulfonic acid (2.90 mmol, 0.50 g) is added, the temperature is raised to 90° C., and the mixture is stirred for 3 h. The resulting brown reaction mixture is then dissolved in 500 mL of diethyl ether. The ether solution is washed with water (3 portions of 500 mL) to eliminate any unreacted para-formaldehyde, 4-nonylphenol or allyl amine and dried over sodium sulfate. The solvent is evaporated under reduced pressure and the product is dried in a vacuum oven.

¹H-NMR (300 MHz, CDCl₃): δ (ppm)=6.89 (1 H, d), 6.82 (1 H, s), 6.55 (1 H,), 5.76 (1 H, m), 5.01 (2 H, m), 4.66 (2 H, s), 3.85 (2 H, s), 3.21 (2 H, d), 2.99 (2 H, m), 1.91 (2 H, m), 1.13 (2 H, m), 1.08 (2 H, m), 0.99 (4 H, m), 0.67 (4 H, m), 0.53 (3 H, m).

¹³C-NMR (75 MHz, CDCl₃): δ (ppm)=136.7, 126.6, 125.9, 125.7, 119.2, 118.0, 116.4, 82.8, 57.3, 53.0, 36.4, 32.0, 31.5, 29.8, 29.4, 29.1, 22.5, 14.6.

FTIR (ATR): v (cm⁻¹)=2957, 2851, 1500, 1378, 1230, 1122, 988, 822, 820, 748.

### 2.B. Synthesis of Homopolymers (Comparative Examples)

### 2.B1. Homopolymerization of Crosslinked Benzoxazines (Comparative Examples)

The acronym for the crosslinked (Cl) copolymers of the benzoxazine Bₙ and the reactive diluent RD is composed as follows: Cl[p(Bₙ+RD)₁₀₀].

As tetrathiol 4SH, pentaerythritol tetrakis(3-mercaptopropionate) is used.

As photoinitiator PI, ethyl(2,4,6-trimethylbenzoyl)phenylphosphinic ethyl ester is used.

### 2.B1.1. EXAMPLE 1 (COMPARATIVE)

### Crosslinked Copolymers of 6,6′-(propane-2,2-diyl)bis(3-allyl-3,4-dihydro-2H-benzo[e][1,3]oxazine) and 3-allyl-3,4-dihydro-2H-benzo[e][1,3]oxazine

### (Acronym: Cl[p(B₁+RD)₁₀₀]):

Boron trifluoride etherate (1.10 mmol, 156 mg) is added to 4SH (3.1 mmol, 1.51 g). The mixture is added to a liquid blend of B₁ (8.1 mmol, 3.16 g), RD (13.8 mmol, 2.41 g) and PI (0.1 mmol, 31.6 mg). Using UV-light, thiol-ene precuring is performed for 15 min. Subsequent heating up to 100° C. for 24 h results in the cationic ring-opening polymerization of B₁ and RD.

FTIR (ATR): v (cm⁻¹)=3011, 2968, 2919, 1740, 1625, 1438, 1364, 1215, 1117, 911.

### 2.B1.2. EXAMPLE 2 (COMPARATIVE)

### Crosslinked Copolymers of bis(3-allyl-3,4-dihydro-2H-benzo[e][1,3]oxazin-6-yl)methane and 3-allyl-3,4-dihydro-2H-benzo[e][1,3]oxazine

### (Acronym: Cl[p(B₂+RD)₁₀₀]):

Boron trifluoride etherate (1.10 mmol, 156.1 mg) is added to 4SH (3.1 mmol, 1.51 g). The mixture is added to a liquid blend of B₂ (8.1 mmol, 2.94 g), RD (10.3 mmol, 1.80 g) and PI (0.1 mmol, 31.6 mg). Using UV-light, thiol-ene precuring is performed for 15 min. Subsequent heating up to 100° C. for 24 h results in the cationic ring-opening polymerization of B₂ and RD.

FTIR (ATR): v (cm⁻¹)=3017, 2968, 2860, 1740, 1440, 1368, 1226, 1054, 869.

### 2.B1.3. EXAMPLE 3 (Comparative)

### Crosslinked copolymers of 6,6′-sulfonylbis(3-allyl-3,4-dihydro-2H-benzo[e][1,3]oxazine) and 3-allyl-3,4-dihydro-2H-benzo[e][1,3]-oxazine

### (Acronym: Cl[p(B3+RD)₁₀₀]):

Boron trifluoride etherate (1.10 mmol, 156.1 mg) is added to 4SH (3.1 mmol, 1.51 g). The mixture is added to a liquid blend of B3 (8.1 mmol, 3.34 g), RD (10.3 mmol, 1.80 g) and PI (0.1 mmol, 31.6 mg). Using UV-light, thiol-ene precuring is performed for 15 min. Subsequent heating up to 100° C. for 24 h results in the cationic ring-opening polymerization of B₃ and RD.

FTIR (ATR): v (cm⁻¹)=2921, 2853, 1683, 1578, 1458, 1290, 1098, 911

### 2.B1.4. EXAMPLE 4 (COMPARATIVE)

### Crosslinked Copolymers of 2,9-diallyl-1,2,3,8,9,10-hexahydrobenzo[2,1-e:3,4-e′]bis([1,3]oxazine and 3-allyl-3,4-dihydro-2H-benzo[e][1,3]oxazine

### (acronym: Cl[p(B₄+RD)₁₀₀]):

Boron trifluoride etherate (1.10 mmol, 156.1 mg) is added to 4SH (3.1 mmol, 1.51 g). The mixture is added to a liquid blend of B4 (12.1 mmol, 3.29 g), RD (9.8 mmol, 2.66 g) and PI (0.1 mmol, 31.6 mg). Using UV-light, thiol-ene precuring is performed for 15 min. Subsequent heating up to 100° C. for 24 h results in the cationic ring-opening polymerization of B4 and RD.

FTIR (ATR): v (cm⁻¹)=3017, 2970, 1740, 1640, 1444, 1368, 1219, 911.

2.B2. Homopolymerization of Cyclic Carbonates (comparative examples)

### 2.B2.1. EXAMPLE 5 (comparative): Poly(ethylene carbonate)

### (Acronym: pEC):

Boron trifluoride etherate (1.86 mmol, 265 mg) is added to molten ethylene carbonate (EC) (0.10 mol, 8.8 g). The reaction mixture is heated up to 100° C. overnight without stirring. After 24 h, a solid polymer is obtained.

FTIR (ATR): v (cm⁻¹)=3007, 2970, 2878, 1744, 1442, 1364, 1219, 1048, 879

2.B2.2. EXAMPLE 6 (COMPARATIVE)

### Poly(Propylene Carbonate)

### (Acronym: pPC):

Boron trifluoride etherate (1.9 mmol, 265 mg) is added to propylene carbonate (PC) (0.10 mol, 10.2 g). The reaction mixture is heated up to 100° C. overnight without stirring. After 24 h, a solid polymer is obtained.

FTIR (ATR): v (cm⁻¹)=2988, 2923, 1789, 1485, 1379, 1180, 1040, 885, 773.

2.C. Copolymer Synthesis (Examples according to the invention)

### 2.C.1. EXAMPLE 7 (INVENTION)

### Cl[p(B₁+RD)ᵢ-stat-pEC₁₀₀₋ᵢ] (i=99, 97, 93, 86, 70, 30, 1) & [p(B₁+RD)ᵢ-stat-pEC₁₀₀₋ᵢ](i=86)

Boron trifluoride etherate (amount: A) is added to 4SH (amount: B). The mixture is added to a liquid blend of B₁ (amount: C), RD (amount: D), EC (amount: E) and PI (amount: F)—see Table 1 below. Using UV-light, thiol-ene precuring is performed for 15 min. Subsequent heating up to 100° C. for 24 h results in the cationic ring-opening copolymerization of B₁, RD and EC. In the case of [p(B₁+RD)₈₆-stat-pEC₁₄], neither 4SH nor PI were added; no UV illumination was applied.

FTIR (ATR): v (cm⁻¹)=3074, 2964, 2825, 1809, 1593, 1493, 1344, 1222, 1120, 1073, 922, 816, 754.

TABLE 1

| | A | B | C | D | E | F |
|---|---|---|---|---|---|---|
| Identifier | [mmol//g] | [mmol//g] | [mmol//g] | [mmol//g] | [mmol//g] | [mmol//g] |
| Cl[p(B₁ + RD)₉₉-stat-pEC₁] | 1.10//0.156 | 3.1//1.51 | 8.1//3.16 | 13.8//2.41 | 0.83//0.073 | 0.10//0.031 |
| Cl[p(B₁ + RD)₉₇-stat-pEC₃] | 1.10//0.156 | 3.1//1.51 | 8.1//3.16 | 13.8//2.41 | 2.55//0.225 | 0.10//0.031 |
| Cl[p(B₁ + RD)₉₃-stat-pEC₇] | 1.10//0.156 | 3.1//1.51 | 8.1//3.16 | 13.8//2.41 | 6.61//0.550 | 0.10//0.031 |
| Cl[p(B₁ + RD)₈₆-stat-pEC₁₄] | 1.10//0.156 | 3.1//1.51 | 8.1//3.16 | 13.8//2.41 | 15.7//1.38 | 0.10//0.031 |
| Cl[p(B₁ + RD)₇₀-stat-pEC₃₀] | 1.10//0.156 | 3.1//1.51 | 8.1//3.16 | 13.8//2.41 | 38.5//3.11 | 0.10//0.031 |
| Cl[p(B₁ + RD)₃₀-stat-pEC₇₀] | 0.20//0.029 | 0.57//0.280 | 1.48//0.58 | 2.52//0.44 | 38.5//3.11 | 0.018//0.0060 |
| Cl[p(B₁ + RD)₁-stat-pEC₉₉] | 0.03//0.0042 | 0.084//0.041 | 0.22//0.085 | 0.37//0.065 | 231//18.66 | 0.003//0.0011 |
| [p(B₁ + RD)₈₆-stat-pEC₁₄] | 1.10//0.156 | 0//0 | 8.1//3.16 | 13.8//2.41 | 15.7//1.38 | 0//0 |

2.C.2. EXAMPLE 8 (INVENTION)

### Cl[p(B₁+RD)ᵢ-stat-pPC₁₀₀₋ᵢ] (i=99, 97, 93, 86, 70, 30, 1) & [p(B₁+RD)ᵢ-stat-pPC₁₀₀₋ᵢ] (i=86)

Boron trifluoride etherate (amount: A) is added to 4SH (amount: B). The mixture is added to a liquid blend of B₁ (amount: C), RD (amount: D), PC (amount: E) and PI (amount: F)—see Table 2 below. Using UV-light, thiol-ene precuring is performed for 15 min. Subsequent heating up to 100° C. for 24 h results in the cationic ring-opening copolymerization of B1, RD, and PC. In the case of [p(B₁+RD)₈₆-stat-pPC₁₄], neither 4SH nor PI were added; no UV illumination was applied.

FTIR (ATR): v (cm⁻¹)=2968, 2925, 2821, 1740, 1591, 1493, 1366, 1236, 1132, 987, 911, 820, 754.

TABLE 2

| | A | B | C | D | E | F |
|---|---|---|---|---|---|---|
| Identifier | [mmol//g] | [mmol//g] | [mmol//g] | [mmol//g] | [mmol//g] | [mmol//g] |
| Cl[p(B₁ + RD)₉₉-stat-pPC₁] | 1.10//0.156 | 3.1//1.51 | 8.1//3.16 | 13.8//2.41 | 0.72//0.0734 | 0.100//0.0310 |
| Cl[p(B₁ + RD)₉₇-stat-pPC₃] | 1.10//0.156 | 3.1//1.51 | 8.1//3.16 | 13.8//2.41 | 2.2//0.225 | 0.100//0.0310 |
| Cl[p(B₁ + RD)₉₃-stat-pPC₇] | 1.10//0.156 | 3.1//1.51 | 8.1//3.16 | 13.8//2.41 | 5.4//0.55 | 0.100//0.0310 |
| Cl[p(B₁ + RD)₈₆-stat-pPC₁₄] | 1.10//0.156 | 3.1//1.51 | 8.1//3.16 | 13.8//2.41 | 13.5//1.38 | 0.100//0.0310 |
| Cl[p(B₁ + RD)₇₀-stat-pPC₃₀] | 1.10//0.156 | 3.1//1.51 | 8.1//3.16 | 13.8//2.41 | 30.5//3.11 | 0.100//0.0310 |
| Cl[p(B₁ + RD)₃₀-stat-pPC₇₀] | 0.20//0.0285 | 0.57//0.280 | 1.48//0.578 | 2.52//0.441 | 30.5//3.11 | 0.018//0.0060 |
| Cl[p(B₁ + RD)₁-stat-pPC₉₉] | 0.03//0.0042 | 0.084//0.041 | 0.22//0.085 | 0.371//0.065 | 183//18.66 | 0.003//0.0011 |
| [p(B₁ + RD)₈₆-stat-pPC₁₄] | 1.10//0.156 | 0//0 | 8.1//3.16 | 13.8//2.41 | 13.5//1.38 | 0//0 |

### 2.C.3. EXAMPLE 9 (INVENTION)

### Cl[p(B₂₊RD)ᵢstat-pEC₁₀₀₋ᵢ] (i=99, 97, 93, 86, 70, 30, 1) & [p(B₂₊RD)ᵢ-stat-pEC₁₀₀₋ᵢ] (i=86)

Boron trifluoride etherate (amount: A) is added to 4SH (amount: B). The mixture is added to a liquid blend of B2 (amount: C), RD (amount: D), EC (amount: E) and PI (amount: F)—see Table 3 below. Using UV-light, thiol-ene precuring is performed for 15 min. Subsequent heating up to 100° C. for 24 h results in the cationic ring-opening polymerization of B₂, RD, and EC. In the case of [p(B₂₊RD)86-stat-pEC₁₄], neither 4SH nor PI were added; no UV illumination was applied.

FTIR (ATR): v (cm⁻¹)=3005, 2968, 2926, 1736, 1594, 1438, 1364, 1230, 1236, 987, 920, 811.

TABLE 3

| | A | B | C | D | E | F |
|---|---|---|---|---|---|---|
| Identifier | [mmol//g] | [mmol//g] | [mmol//g] | [mmol//g] | [mmol//g] | [mmol//g] |
| Cl[p(B₂ + RD)₉₉-stat-pEC₁] | 1.10//0.156 | 3.1//1.51 | 8.1//2.94 | 10.3//1.80 | 0.74//0.0652 | 0.100//0.031 |
| Cl[p(B₂ + RD)₉₇-stat-pEC₃] | 1.10//0.156 | 3.1//1.51 | 8.1//2.94 | 10.3//1.80 | 2.26//0.199 | 0.100//0.031 |
| Cl[p(B₂ + RD)₉₃-stat-pEC₇] | 1.10//0.156 | 3.1//1.51 | 8.1//2.94 | 10.3//1.80 | 5.5//0.484 | 0.100//0.031 |
| Cl[p(B₂ + RD)₈₆-stat-pEC₁₄] | 1.10//0.156 | 3.1//1.51 | 8.1//2.94 | 10.3//1.80 | 13.9//1.23 | 0.100//0.031 |
| Cl[p(B₂ + RD)₇₀-stat-pEC₃₀] | 1.10//0.156 | 3.1//1.51 | 8.1//2.94 | 10.3//1.80 | 31.3//2.76 | 0.100//0.031 |
| Cl[p(B₂ + RD)₃₀-stat-pEC₇₀] | 0.17//0.0247 | 0.49//0.239 | 1.28//0.466 | 1.62//0.285 | 31.3//2.76 | 0.015//0.0016 |
| Cl[p(B₂ + RD)₁-stat-pEC₉₉] | 0.025//0.0035 | 0.070//0.0339 | 0.182//0.0661 | 0.23//0.0405 | 188//16.6 | 0.002//0.0007 |
| [p(B₂ + RD)₈₆-stat-pEC₁₄] | 1.10//0.156 | 0//0 | 8.1//2.94 | 10.3//1.80 | 13.9//1.23 | 0//0 |

### 2.C.4. EXAMPLE 10 (INVENTION)

### Cl[p(B₂₊RD)ᵢ-stat-pPC₁₀₀₋ᵢ] (i=99, 97, 93, 86, 70, 30, 1) & [p(B₂+RD)ᵢ-stat-pPC₁₀₀₋ᵢ] (i=86)

Boron trifluoride etherate (amount: A) is added to 4SH (amount: B). The mixture is added to a liquid blend of B2 (amount: C), RD (amount: D), PC (amount: E) and PI (amount: F)—see Table 4 below. Using UV-light, thiol-ene precuring is performed for 15 min. Subsequent heating up to 100° C. for 24 h results in the cationic ring-opening polymerization of B₂, RD, and PC. In the case of [p(B₂+RD)86-stat-pPC₁₄], neither 4SH nor PI were added; no UV illumination was applied.

FTIR (ATR): v (cm^{—1})=3070, 2923, 2839, 1797, 1736, 1591, 1493, 1354, 1246, 1117, 991, 926, 765.

TABLE 4

| | A | B | C | D | E | F |
|---|---|---|---|---|---|---|
| Identifier | [mmol//g] | [mmol//g] | [mmol//g] | [mmol//g] | [mmol//g] | [mmol//g] |
| Cl[p(B₂ + RD)₉₉-stat-pPC₁] | 1.10//0.156 | 3.1//1.51 | 8.1//2.94 | 10.3//1.80 | 0.64//0.0652 | 0.100//0.031 |
| Cl[p(B₂ + RD)₉₇-stat-pPC₃] | 1.10//0.156 | 3.1//1.51 | 8.1//2.94 | 10.3//1.80 | 1.95//0.199 | 0.100//0.031 |
| Cl[p(B₂ + RD)₉₃-stat-pPC₇] | 1.10//0.156 | 3.1//1.51 | 8.1//2.94 | 10.3//1.80 | 4.8//0.484 | 0.100//0.031 |
| Cl[p(B₂ + RD)₈₆-stat-pPC₁₄] | 1.10//0.156 | 3.1//1.51 | 8.1//2.94 | 10.3//1.80 | 12//1.23 | 0.100//0.031 |
| Cl[p(B₂ + RD)₇₀-stat-pPC₃₀] | 1.10//0.156 | 3.1//1.51 | 8.1//2.94 | 10.3//1.80 | 27//2.76 | 0.100//0.031 |
| Cl[p(B₂ + RD)₃₀-stat-pPC₇₀] | 0.17//0.0247 | 0.49//0.239 | 1.28//0.466 | 1.62//0.285 | 27//2.76 | 0.015//0.0016 |
| Cl[p(B₂ + RD)₁-stat-pPC₉₉] | 0.025//0.0035 | 0.070//0.0339 | 0.182//0.0661 | 0.23//0.0405 | 162//16.6 | 0.002//0.0007 |
| [p(B₂ + RD)₈₆-stat-pPC₁₄] | 1.10//0.156 | 0//0 | 8.1//2.94 | 10.3//1.80 | 12//1.23 | 0//0 |

### 2.C.5. EXAMPLE 11 (INVENTION)

### Cl[p(B₃₊RD)ᵢ-stat-pEC₁₀₀₋ᵢ]=(i=99, 97, 93, 86, 70, 30, 1)

Boron trifluoride etherate (amount: A) is added to 4SH (amount: B). The mixture is added to a liquid blend of B3 (amount: C), RD (amount: D), EC (amount: E) and PI (amount: F)—see Table 5 below. Using UV-light, thiol-ene precuring is performed for 15 min. Subsequent heating up to 100° C. for 24 h results in the cationic ring-opening polymerization of B₃, RD, and EC.

FTIR (ATR): v (cm⁻¹)=3004, 2919, 2815, 1729, 1592, 1452, 1252, 1136, 999, 920.

TABLE 5

| | A | B | C | D | E | F |
|---|---|---|---|---|---|---|
| Identifier | [mmol//g] | [mmol//g] | [mmol//g] | [mmol//g] | [mmol//g] | [mmol//g] |
| Cl[p(B₃ + RD)₉₉-stat-pEC₁] | 1.10//0.156 | 3.1//1.51 | 8.1//3.34 | 10.3//1.80 | 0.78//0.0691 | 0.100//0.0310 |
| Cl[p(B₃ + RD)₉₇-stat-pEC₃] | 1.10//0.156 | 3.1//1.51 | 8.1//3.34 | 10.3//1.80 | 2.4//0.212 | 0.100//0.0310 |
| Cl[p(B₃ + RD)₉₃-stat-pEC₇] | 1.10//0.156 | 3.1//1.51 | 8.1//3.34 | 10.3//1.80 | 5.9//0.515 | 0.100//0.0310 |
| Cl[p(B₃ + RD)₈₆-stat-pEC₁₄] | 1.10//0.156 | 3.1//1.51 | 8.1//3.34 | 10.3//1.80 | 12.6/1.11 | 0.100//0.0310 |
| Cl[p(B₃ + RD)₇₀-stat-pEC₃₀] | 1.10//0.156 | 3.1//1.51 | 8.1//3.34 | 10.3//1.80 | 33.3//2.93 | 0.100//0.0310 |
| Cl[p(B₃ + RD)₃₀-stat-pEC₇₀] | 0.19//0.0270 | 0.54//0.261 | 1.40//0.576 | 1.8//0.311 | 33.3//2.93 | 0.017//0.0054 |
| Cl[p(B₃ + RD)₁-stat-pEC₉₉] | 0.027//0.0038 | 0.076//0.0370 | 0.198//0.0818 | 0.25//0.0447 | 188//16.6 | 0.002//0.0008 |

### 2.C.6. EXAMPLE 12 (INVENTION)

### Cl[p(B3+RD)ᵢ-stat-pPC₁₀₀₋ᵢ] (i=99, 97, 93, 86, 70, 30, 1)

Boron trifluoride etherate (amount: A) is added to 4SH (amount: B). The mixture is added to a liquid blend of B₃ (amount: C), RD (amount: D), PC (amount: E) and PI (amount: F)—see Table 6 below. Using UV-light, thiol-ene precuring is performed for 15 min. Subsequent heating up to 100° C. for 24 h results in the cationic ring-opening polymerization of B₃, RD, and PC.

FTIR (ATR): v (cm⁻)=3076, 2925, 2815, 1730, 1595, 1460, 1256, 1142, 971, 916, 756.

TABLE 6

| | A | B | C | D | E | F |
|---|---|---|---|---|---|---|
| Identifier | [mmol//g] | [mmol//g] | [mmol//g] | [mmol//g] | [mmol//g] | [mmol//g] |
| Cl[p(B₃ + RD)₉₉-stat-pPC₁] | 1.10//0.156 | 3.1//1.51 | 8.1//3.34 | 10.3//1.80 | 0.68//0.0691 | 0.100//0.0310 |
| Cl[p(B₃ + RD)₉₇-stat-pPC₃] | 1.10//0.156 | 3.1//1.51 | 8.1//3.34 | 10.3//1.80 | 2.07//0.212 | 0.100//0.0310 |
| Cl[p(B₃ + RD)₉₃-stat-pPC₇] | 1.10//0.156 | 3.1//1.51 | 8.1//3.34 | 10.3//1.80 | 5.0//0.515 | 0.100//0.0310 |
| Cl[p(B₃ + RD)₈₆-stat-pPC₁₄] | 1.10//0.156 | 3.1//1.51 | 8.1//3.34 | 10.3//1.80 | 10.9/1.11 | 0.100//0.0310 |
| Cl[p(B₃ + RD)₇₀-stat-pPC₃₀] | 1.10//0.156 | 3.1//1.51 | 8.1//3.34 | 10.3//1.80 | 28.7//2.93 | 0.100//0.0310 |
| Cl[(pB₃ + RD)₃₀-stat-pPC₇₀] | 0.19//0.0270 | 0.54//0.261 | 1.40//0.577 | 1.77//0.311 | 28.7//2.93 | 0.017//0.0054 |
| Cl[(pB₃ + RD)₁-stat-pPC₉₉] | 0.027//0.0038 | 0.076//0.0370 | 0.198//0.0818 | 0.251//0.441 | 162//16.6 | 0.002//0.0008 |

### 2.C.7. EXAMPLE 13 (INVENTION)

### Cl[p(B4+RD)ᵢ-stat-pEC₁₀₀₋ᵢ](i=99, 97, 93, 86, 70, 30, 1)

Boron trifluoride etherate (amount: A) is added to 4SH (amount: B). The mixture is added to a liquid blend of B4 (amount: C), RD (amount: D), EC (amount: E) and PI (amount: F)—see Table 7 below. Using UV-light, thiol-ene precuring is performed for 15 min. Subsequent heating up to 100° C. for 24 h results in the cationic ring-opening polymerization of B₄, RD, and EC.

FTIR (ATR): v (cm⁻¹)=2998, 2917, 1762, 1638, 1622, 1479, 1391, 1156, 1052, 969, 716.

TABLE 7

| | A | B | C | D | E | F |
|---|---|---|---|---|---|---|
| Identifier | [mmol//g] | [mmol//g] | [mmol//g] | [mmol//g] | [mmol//g] | [mmol//g] |
| Cl[p(B₄ + RD)₉₉-stat-pEC₁] | 1.10//0.156 | 3.1//1.51 | 12.1//3.29 | 9.8//2.66 | 0.88//0.0773 | 0.100//0.0310 |
| Cl[p(B₄ + RD)₉₇-stat-pEC₃] | 1.10//0.156 | 3.1//1.51 | 12.1//3.29 | 9.8//2.66 | 2.69//0.237 | 0.100//0.0310 |
| Cl[p(B₄ + RD)₉₃-stat-pEC₇] | 1.10//0.156 | 3.1//1.51 | 12.1//3.29 | 9.8//2.66 | 6.5//0.576 | 0.100//0.0310 |
| Cl[p(B₄ + RD)₈₆-stat-pEC₁₄] | 1.10//0.156 | 3.1//1.51 | 12.1//3.29 | 9.8//2.66 | 16.5//1.46 | 0.100//0.0310 |
| Cl[(pB₄ + RD)₇₀-stat-pEC₃₀] | 1.10//0.156 | 3.1//1.51 | 12.1//3.29 | 9.8//2.66 | 37.2//3.28 | 0.100//0.0310 |
| Cl[(pB₄ + RD)₃₀-stat-pEC₇₀] | 0.203//0.0288 | 0.57//0.278 | 2.23//0.607 | 1.80//0.491 | 37.2//3.28 | 0.018//0.0057 |
| Cl[(pB₄ + RD)₁-stat-pEC₉₉] | 0.029//0.0041 | 0.081//0.0393 | 0.315//0.0856 | 0.254//0.0692 | 223//19.7 | 0.003//0.0008 |

### 2.C.8. EXAMPLE 14 (INVENTION)

### Cl[p(B₄₊RD)ᵢ-stat-pPC_{100-i] (i=}99, 97, 93, 86, 70, 30, 1)

Boron trifluoride etherate (amount: A) is added to 4SH (amount:B). The mixture is added to a liquid blend of B₄ (amount: C), RD (amount: D), PC (amount: E) and PI (amount: F)—see Table 8 below. Using UV-light, thiol-ene precuring is performed for 15 min. Subsequent heating up to 100° C. for 24 h results in the cationic ring-opening polymerization of B₄, RD, and PC.

FTIR (ATR): v (cm⁻¹)=3070, 2928, 2854, 1740, 1640, 1617, 1436, 1354, 1217, 1130, 936.

TABLE 8

| | A | B | C | D | E | F |
|---|---|---|---|---|---|---|
| Identifier | [mmol//g] | [mmol//g] | [mmol//g] | [mmol//g] | [mmol//g] | [mmol//g] |
| Cl[p(B₄ + RD)₉₉-stat-pPC₁] | 1.10//0.156 | 3.1//1.51 | 12.1//3.29 | 9.8//2.66 | 0.76//0.0773 | 0.100//0.0310 |
| Cl[p(B₄ + RD)₉₇-stat-pPC₃] | 1.10//0.156 | 3.1//1.51 | 12.1//3.29 | 9.8//2.66 | 2.32//0.237 | 0.100//0.0310 |
| Cl[p(B₄ + RD)₉₃-stat-pPC₇] | 1.10//0.156 | 3.1//1.51 | 12.1//3.29 | 9.8//2.66 | 5.6//0.576 | 0.100//0.0310 |
| Cl[p(B₄ + RD)₈₆-stat-pPC₁₄] | 1.10//0.156 | 3.1//1.51 | 12.1//3.29 | 9.8//2.66 | 14.3//1.46 | 0.100//0.0310 |
| Cl[p(B₄ + RD)₇₀-stat-pPC₃₀] | 1.10//0.156 | 3.1//1.51 | 12.1//3.29 | 9.8//2.66 | 32.1//3.28 | 0.100//0.0310 |
| Cl[p(B₄ + RD)₃₀-stat-pPC₇₀] | 0.203//0.0288 | 0.57//0.278 | 2.23//0.607 | 1.80//0.491 | 32.1//3.28 | 0.018//0.0057 |
| Cl[p(B₄ + RD)₁-stat-pPC₉₉] | 0.029//0.0041 | 0.081//0.0393 | 0.32//0.0856 | 0.254//0.0692 | 193//19.7 | 0.003//0.0008 |

### 3. Expansion

3.A. Visual presentation of a copolymer according to the invention

In order to visually demonstrate the effect of the expansion upon curing, specimens were prepared as follows: Boron trifluoride etherate (1.1 mmol, 0.156 g) was added to 4SH (3.1 mmol, 1.51 g). The mixture was added to a liquid blend of B₁ (8.1 mmol, 3.16 g), RD (13.8 mmol, 2.41 g), PC (15.7 mmol, 1.38 g) and PI (0.10 mmol, 0.031 g) and poured into a circular-shaped mold. Using UV-light, thiol-ene precuring was performed for 15 min. The specimen was subsequently divided into two parts. One part was heated up to 100° C. for 24 h to enable cationic ring-opening copolymerization of B₁, RD, and PC; the other part was kept without additional curing.

FIG. 1 illustrates the thiol-ene precured but not copolymerized specimen (smaller part on the right) and the thiol-ene precured and copolymerized specimen (larger part on the left).

3.B. Expansion rates

Expansion rates were quantified by density measurements of the homopolymers (see above comparative examples 1-6) on the one hand and the compolymers according to the invention (see above examples 7-14 according to the invention) on the other. Expansion rates were measured and calculated as described above under chapter 1.A.2 Methods. The expansion rates are given in Tables 10-17 below in chapters 3.B.1. to 3.B.8. and are further illustrated in accompanying FIGS. 2-9.

While the comparative examples have been included within the individual tables in chapters 3.B.1. to 3.B.8., the NON-copolymerized congeners, namely the polybenzoxazine homopolymers on the one hand as well the poly(cyclic carbonate) homopolymers on the other, have been listed in Table 9 hereinafter in order to provide an overview of the expansion rates achievable according to the current state-of-the-art:

TABLE 9

| Identifier | Expansion rate [%] |
|---|---|
| Cl[p(B₁ + RD)₁₀₀] | 3.3 |
| Cl[p(B₂ + RD)₁₀₀] | 2.1 |
| Cl[p(B₃ + RD)₁₀₀] | 1.0 |
| Cl[p(B₄ + RD)₁₀₀] | 0.2 |
| pEC | −0.8 |
| pPC | −0.5 |

3.B.1. Cl[p(B₁+RD)ᵢ-stat-pEC₁₀₀₋ᵢ] (i=100, 99, 97, 93, 86, 70, 30, 1, 0) & [p(B₁+RD)ᵢ-stat-pEC₁₀₀₋ᵢ] (i=86)

TABLE 10

| | | δ_{Monomer} | δ_{Polymer} | Expansion |
|---|---|---|---|---|
| Identifier | Appearance | [g × cm⁻¹] | [g × cm⁻¹] | rate [%] |
| Cl[p(B₁ + RD)₁₀₀] | solid, brittle | 1.169 | 1.132 | 3.3 |
| Cl[p(B₁ + RD)₉₉-stat-pEC₁] | solid, brittle | 1.174 | 1.129 | 4 |
| Cl[p(B₁ + RD)₉₇-stat-pEC₃] | solid, soft | 1.182 | 1.059 | 11.6 |
| Cl[p(B₁ + RD)₉₃-stat-pEC₇] | solid, soft | 1.117 | 0.989 | 12.9 |
| Cl[p(B₁ + RD)₈₆-stat-pEC₁₄] | rubbery | 1.143 | 0.938 | 21.9 |
| Cl[p(B₁ + RD)₇₀-stat-pEC₃₀] | rubbery | 1.273 | 0.962 | 32.4 |
| Cl[p(B₁ + RD)₃₀-stat-pEC₇₀] | rubbery, swollen | 1.217 | 1.182 | 2.9 |
| Cl[p(B₁ + RD)₁-stat-pEC₉₉] | liquid | 1.264 | 1.264 | 0 |
| pEC | liquid, highly viscous | 1.311 | 1.321 | −0.8 |
| [p(B₁ + RD)₈₆-stat-pEC₁₄] | rubbery | 1.187 | 1.014 | 17.1 |

FIG. 2 shows the expansion rate in [%] as a function of the concentration of the cyclic carbonate in the copolymer Cl[p(B₁+RD)₀₋₁₀₀-stat-pEC₀₋₁₀₀].

3.B.2. Cl[p(B₁+RD)ᵢ-stat-pPC₁₀₀₋ᵢ] (i=100, 99, 97, 93, 86, 70, 30, 1, 0) & [p(B₁+RD)ᵢ-stat-pPC₁₀₀₋ᵢ] (i=86)

TABLE 11

| | | δ_{Monomer} | δ_{Polymer} | Expansion |
|---|---|---|---|---|
| Identifier | Appearance | [g × cm⁻¹] | [g × cm⁻¹] | rate [%] |
| Cl[p(B₁ + RD)₁₀₀] | solid, brittle | 1.169 | 1.132 | 3.3 |
| Cl[p(B₁ + RD)₉₉-stat-pPC₁] | solid, brittle | 1.173 | 1.149 | 2.1 |
| Cl[p(B₁ + RD)₉₇-stat-pPC₃] | solid, soft | 1.184 | 1.089 | 8.8 |
| Cl[p(B₁ + RD)₉₃-stat-pPC₇] | solid, soft | 1.174 | 1.003 | 17.1 |
| Cl[p(B₁ + RD)₈₆-stat-pPC₁₄] | rubbery | 1.172 | 0.944 | 24.1 |
| Cl[p(B₁ + RD)₇₀-stat-pPC₃₀] | rubbery | 1.232 | 0.936 | 31.7 |
| Cl[p(B₁ + RD)₃₀-stat-pPC₇₀] | rubbery, swollen | 1.199 | 1.176 | 2 |
| Cl[p(B₁ + RD)₁-stat-pPC₉₉] | liquid | 1.226 | 1.225 | 0.1 |
| pPC | liquid, highly viscous | 1.229 | 1.235 | −0.5 |
| [p(B₁ + RD)₈₆-stat-pPC₁₄] | rubbery | 1.202 | 1.027 | 17 |

FIG. 3 shows the expansion rate in [%] as a function of the concentration of the cyclic carbonate in the copolymer Cl[p(B₁+RD)₀₋₁₀₀-stat-pPC₀₋₁₀₀].

3.B.3. Cl[p(B₂₊RD)-stat-pEC₁₀₀₋ᵢ] (i=100, 99, 97, 93, 86, 70, 30, 1, 0) & [p(B2+RD)ᵢ-stat-pEC₁₀₀₋ᵢ] (i=86)

TABLE 12

| | | δ_{Monomer} | δ_{Polymer} | Expansion |
|---|---|---|---|---|
| Identifier | Appearance | [g × cm⁻¹] | [g × cm⁻¹] | rate [%] |
| Cl[p(B₂ + RD)₁₀₀] | solid, brittle | 1.152 | 1.128 | 2.1 |
| Cl[p(B₂ + RD)₉₉-stat-pEC₁] | solid, soft | 1.161 | 1.129 | 2.9 |
| Cl[p(B₂ + RD)₉₇-stat-pEC₃] | solid, soft | 1.172 | 1.059 | 10.7 |
| Cl[p(B₂ + RD)₉₃-stat-pEC₇] | solid, soft | 1.192 | 0.989 | 20.5 |
| Cl[p(B₂ + RD)₈₆-stat-pEC₁₄] | rubbery | 1.208 | 0.938 | 28.9 |
| Cl[p(B₂ + RD)₇₀-stat-pEC₃₀] | rubbery | 1.219 | 0.962 | 26.7 |
| Cl[p(B₂ + RD)₃₀-stat-pEC₇₀] | rubbery, swollen | 1.231 | 1.182 | 4.1 |
| Cl[p(B₂ + RD)₁-stat-pEC₉₉] | liquid | 1.266 | 1.264 | 0.1 |
| pEC | liquid, highly viscous | 1.311 | 1.321 | −0.8 |
| [p(B₂ + RD)₈₆-stat-pEC₁₄] | rubbery | 1.191 | 1.036 | 15 |

FIG. 4 shows the expansion rate in [%] as a function of the concentration of the cyclic carbonate in the copolymer Cl[p(B₂+RD)₀₋₁₀₀-stat-pEC₀₋₁₀₀].

3.B.4. Cl[p(B₂+RD)ᵢ-stat-pPC₁₀₀₋ᵢ] (i=100, 99, 97, 93, 86, 70, 30, 1, 0) & [p(B2+RD)ᵢ-stat-pPC₁₀₀₋ᵢ](i=86)

TABLE 13

| | | δ_{Monomer} | δ_{Polymer} | Expansion |
|---|---|---|---|---|
| Identifier | Appearance | [g × cm⁻¹] | [g × cm⁻¹] | rate [%] |
| Cl[p(B₂ + RD)₁₀₀] | solid, brittle | 1.152 | 1.128 | 2.1 |
| Cl[p(B₂ + RD)₉₉-stat-pPC₁] | solid, soft | 1.176 | 1.149 | 2.3 |
| Cl[p(B₂ + RD)₉₇-stat-pPC₃] | solid, soft | 1.183 | 1.089 | 8.6 |
| Cl[p(B₂ + RD)₉₃-stat-pPC₇] | solid, soft | 1.19 | 1.003 | 18.6 |
| Cl[p(B₂ + RD)₈₆-stat-pPC₁₄] | rubbery | 1.201 | 0.944 | 27.2 |
| Cl[p(B₂ + RD)₇₀-stat-pPC₃₀] | rubbery | 1.209 | 0.936 | 29.2 |
| Cl[p(B₂ + RD)₃₀-stat-pPC₇₀] | rubbery, swollen | 1.212 | 1.176 | 3.0 |
| Cl[p(B₂ + RD)₁-stat-pPC₉₉] | liquid | 1.225 | 1.225 | 0 |
| pPC | liquid, highly viscous | 1.229 | 1.235 | −0.5 |
| [p(B₂ + RD)₈₆-stat-pPC₁₄] | rubbery | 1.18 | 1.038 | 13.7 |

FIG. 5 shows the expansion rate in [%] as a function of the concentration of the cyclic carbonate in the copolymer Cl[p(B₂+RD)₀₋₁₀₀-stat-pPC₀₋₁₀₀].

3.B.5. Cl[p(B₃+RD)ᵢ-stat-pEC₁₀₀₋ᵢ] (i=100, 99, 97, 93, 86, 70, 30, 1, 0)

TABLE 14

| | | δ_{Monomer} | δ_{Polymer} | Expansion |
|---|---|---|---|---|
| Identifier | Appearance | [g × cm⁻¹] | [g × cm⁻¹] | rate [%] |
| Cl[p(B₃ + RD)₁₀₀] | solid, brittle | 1.169 | 1.158 | 1.0 |
| Cl[p(B₃ + RD)₉₉-stat-pEC₁] | solid, soft | 1.171 | 1.146 | 2.1 |
| Cl[p(B₃ + RD)₉₇-stat-pEC₃] | solid, soft | 1.179 | 1.124 | 4.9 |
| Cl[p(B₃ + RD)₉₃-stat-pEC₇] | solid, soft | 1.188 | 1.07 | 11 |
| Cl[p(B₃ + RD)₈₆-stat-pEC₁₄] | rubbery | 1.199 | 1.061 | 13 |
| Cl[p(B₃ + RD)₇₀-stat-pEC₃₀] | rubbery | 1.205 | 1.072 | 12.4 |
| Cl[p(B₃ + RD)₃₀-stat-pEC₇₀] | rubbery, swollen | 1.226 | 1.181 | 3.8 |
| Cl[p(B₃ + RD)₁-stat-pEC₉₉] | liquid | 1.25 | 1.251 | −0.1 |
| pEC | liquid, highly viscous | 1.311 | 1.321 | −0.8 |

FIG. 6 shows the expansion rate in [%] as a function of the concentration of the cyclic carbonate in the copolymer Cl[p(B₃+RD)₀₋₁₀₀-stat-pEC₀₋₁₀₀].

3.B.6. Cl[p(B₃+RD)ᵢ-stat-pPC₁₀₀₋ᵢ] (i=100, 99, 97, 93, 86, 70, 30, 1, 0)

TABLE 15

| | | δ_{Monomer} | δ_{Polymer} | Expansion |
|---|---|---|---|---|
| Identifier | Appearance | [g × cm⁻¹] | [g × cm⁻¹] | rate [%] |
| Cl[p(B₃ + RD)₁₀₀] | solid, brittle | 1.169 | 1.158 | 1.0 |
| Cl[p(B₃ + RD)₉₉-stat-pPC₁] | solid, soft | 1.156 | 1.146 | 0.8 |
| Cl[p(B₃ + RD)₉₇-stat-pPC₃] | solid, soft | 1.168 | 1.137 | 2.7 |
| Cl[p(B₃ + RD)₉₃-stat-pPC₇] | solid, soft | 1.177 | 1.105 | 6.6 |
| Cl[p(B₃ + RD)₈₆-stat-pPC₁₄] | rubbery | 1.189 | 1.055 | 12.8 |
| Cl[p(B₃ + RD)₇₀-stat-pPC₃₀] | rubbery | 1.199 | 1.095 | 9.5 |
| Cl[p(B₃ + RD)₃₀-stat-pPC₇₀] | rubbery, swolen | 1.219 | 1.183 | 3.0 |
| Cl[p(B₃ + RD)₁-stat-pPC₉₉] | liquid | 1.226 | 1.231 | −0.4 |
| pPC | liquid, highly viscous | 1.229 | 1.235 | −0.5 |

FIG. 7 shows the expansion rate in [%] as a function of the concentration of the cyclic carbonate in the copolymer Cl[p(B₃+RD)₀₋₁₀₀-stat-pPC₀₋₁₀₀].

3.B.7. Cl[p(B₄+RD)ᵢ-stat-pEC₁₀₀₋ᵢ] (i=100, 99, 97, 93, 86, 70, 30, 1, 0)

TABLE 16

| | | δ_{Monomer} | δ_{Polymer} | Expansion |
|---|---|---|---|---|
| Identifier | Appearance | [g × cm⁻¹] | [g × cm⁻¹] | rate [%] |
| Cl[p(B₄ + RD)₁₀₀] | solid, brittle | 1.207 | 1.204 | 0.2 |
| Cl[p(B₄ + RD)₉₉-stat-pEC₁] | solid, soft | 1.211 | 1.197 | 1.2 |
| Cl[p(B₄ + RD)₉₇-stat-pEC₃] | solid, soft | 1.218 | 1.198 | 1.6 |
| Cl[p(B₄ + RD)₉₃-stat-pEC₇] | rubbery | 1.226 | 1.179 | 4 |
| Cl[p(B₄ + RD)₈₆-stat-pEC₁₄] | rubbery | 1.235 | 1.135 | 8.8 |
| Cl[p(B₄ + RD)₇₀-stat-pEC₃₀] | rubbery | 1.242 | 1.129 | 10 |
| Cl[p(B₄ + RD)₃₀-stat-pEC₇₀] | liquid | 1.247 | 1.179 | 5.8 |
| Cl[p(B₄ + RD)₁-stat-pEC₉₉] | liquid | 1.253 | 1.254 | −0.1 |
| pEC | liquid, highly viscous | 1.311 | 1.321 | −0.8 |

FIG. 8 shows the expansion rate in [%] as a function of the concentration of the cyclic carbonate in the copolymer Cl[p(B₄+RD)₀₋₁₀₀-stat-pEC₀₋₁₀₀].

3.B.8. Cl[p(B₄+RD)ᵢ-stat-pPC₁₀₀₋ᵢ] (i=100, 99, 97, 93, 86, 70, 30, 1, 0)

TABLE 17

| | | δ_{Monomer} | δ_{Polymer} | Expansion |
|---|---|---|---|---|
| Identifier | Appearance | [g × cm⁻¹] | [g × cm⁻¹] | rate [%] |
| Cl[p(B₄ + RD)₁₀₀] | solid, brittle | 1.207 | 1.204 | 0.2 |
| Cl[p(B₄ + RD)₉₉-stat-pPC₁] | solid, soft | 1.202 | 1.187 | 1.2 |
| Cl[p(B₄ + RD)₉₇-stat-pPC₃] | solid, soft | 1.206 | 1.183 | 1.9 |
| Cl[p(B₄ + RD)₉₃-stat-pPC₇] | rubbery | 1.214 | 1.178 | 3.1 |
| Cl[p(B₄ + RD)₈₆-stat-pPC₁₄] | rubbery | 1.231 | 1.182 | 4.1 |
| Cl[p(B₄ + RD)₇₀-stat-pPC₃₀] | rubbery | 1.239 | 1.168 | 6 |
| Cl[p(B₄ + RD)₃₀-stat-pPC₇₀] | liquid | 1.249 | 1.212 | 3 |
| Cl[p(B₄ + RD)₁-stat-pPC₉₉] | liquid | 1.259 | 1.255 | 0.3 |
| pPC | liquid, highly viscous | 1.229 | 1.235 | −0.5 |

FIG. 9 shows the expansion rate in [%] as a function of the concentration of the cyclic carbonate in the copolymer Cl[p(B₄+RD)₀₋₁₀₀-stat-pPC₀₋₁₀₀].

### 3.0 Conclusion

Benzoxazines and cyclic carbonates can be copolymerized according to ring-opening mechanisms. During this curing step, the formulation exhibits volumetric expansion of up to more than 30 vol.-%. Notably, the corresponding homopolymers show volumetric expansion to low extent only (in the case of poly(benzoxazine)s) or no volumetric expansion at all (in the case of poly(cyclic carbonate)s). This behavior of formulations containing benzoxazines and cyclic carbonates is in contrast to numerous other monomer formulations that show shrinkage during the curing step. The extent of volumetric expansion can be tailored in the range from 0 to more than 30 vol.-% by careful choice of the amount and types of benzoxazines and cyclic carbonates. Correspondingly, also the mechanic properties of the corresponding copolymers can be varied from highly brittle to rubber-like types.

The formulation containing the benzoxazines and cyclic carbonates is liquid to semi-solid; its viscosity can be adjusted by the addition of reactive diluents, for example benzoxazine-based reactive diluents. Hence, solvent-free mixtures can be maintained. As such, these formulations can be used as coatings and adhesives that expand during the curing reaction, yielding void-free and crack-free coatings that do not delaminate from the substrate they were adhered to. If the benzoxazines contain additional functional groups such as olefinic units that enable crosslinking by reactions such as the thiol-ene click reaction, the formulation can be pre-cured such that the crosslinking is performed prior to the ring-opening copolymerization. One possible technique for this strategy is the addition of radical photo-initiators and the application of UV irradiation such that crosslinking can be performed at room temperature, while the temperature-inducible ring-opening copolymerization does not yet start. By this strategy, solid specimens with tailor-made geometry can be produced, which can be inserted into cavities, in which they show geometric alignment (e.g., complete filling of the cavity) upon temperature stimuli.

### 4. APPLICATION EXAMPLES

### 4.1. Application Example 1

A precured specimen of the composition Cl[p(B₂+RD)₉₃-stat-pEC₇] and dimensions in the centi- and decimeter range, e.g. 11.8×3.9×0.95 cm, can be used as support and fixation material for windings of electrical machines. This specimen is inserted into a cavity with slightly larger dimensions than those of the specimen, e.g. 12×4×1 cm. Subsequently, heat is applied, and the specimen expands volumetrically due to the ring-opening polymerization. Due to the volumetric expansion, precise geometric alignment of the specimen to the walls of the cavity occurs, which renders excellent stability and, additionally, insulating properties, as no cracks or voids are formed between the specimen and the walls of the cavity.

### 4.2. Application Example 2

A pre-cured specimen of the composition Cl[p(B₂+RD)₈₆-stat-pPC₁₄] and dimensions in the milli-, centi-, or meter range in the form of, e.g., finished bars, sticks, cylinders or spacers, can be used as support and fixation material in high voltage winding insulation barrier systems of electrical equipment, which is liquid or gas insulated. This specimen is inserted into insulation barrier systems with other conventional insulation material. Subsequently, heat is applied during a dry-out process, and the specimen expands volumetrically due to the ring-opening polymerization. Due to the volumetric expansion, precise geometric alignment of the specimen to the surrounding material occurs and compensates the shrinkage and assembling tolerances of those materials, which renders excellent stability and, additionally, insulating properties and form fit support of, e.g., windings or laminated electromagnetic materials.

1. An expandable, polymerizable composition comprising:
at least one benzoxazine and at least one cyclic carbonate, wherein the benzoxazine units comprised in the composition are benzoxazine units which are crosslinkable with each other.

2. The expandable, polymerizable composition according to claim 1, wherein the benzoxazines are crosslinkable by thiol-ene crosslinking.

3. The expandable, polymerizable composition according to claim 1, wherein the crosslinkable benzoxazine is a compound of general Formula (II): 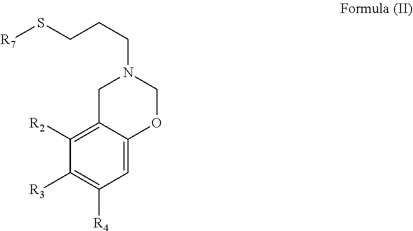
wherein R₂, R₃, and R₄ are each independently H, CH₃, C₂-C₁₅ straight, branched or cyclic alkyl optionally substituted with halogens, alkenyl or alkynyl groups, alkaryl groups, heteroalkyl groups, heteroaryl groups, hydroxy groups, disulfides, sulfonates, ether groups, thioether groups, ester groups, carboxylic acid groups, amine groups, amide groups, azides, or benzoxazines, and R₂ can be linked with R₃ to form a cyclic substituent on the benzene ring or R₃ can be linked with R₄ to form a cyclic substituent on the benzene ring, with the proviso that R₂, R₃, and/or R₄ comprises at least one unsaturated bond; and
R₇ is C₂-C₁₅ straight, branched or cyclic alkyl optionally substituted with halogens, alkaryl groups, heteroalkyl groups, heteroaryl groups, hydroxy groups, disulfides, sulfonates, ether groups, thioether groups, ester groups, carboxylic acid groups, amine groups, amide groups and azides, with the proviso that R₇ comprises at least one thiol group.

4. The expandable, polymerizable composition according to claim 1, wherein the cyclic carbonate is a compound of general Formula (III): 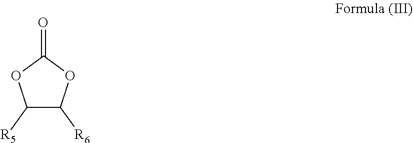
wherein R₅ and R₆ are each independently H, CH₃, C₂-C₁₅ straight, branched or cyclic alkyl optionally substituted with halogens, alkenyl, heteroalkyl groups and hydroxy groups.

5. The expandable, polymerizable composition according to claim 4, wherein the cyclic carbonate is ethylene carbonate or propylene carbonate.

6. The expandable, polymerizable composition according to claim 1, wherein the benzoxazine is derived from a dihydroxybenzene or a bisphenol, preferably selected from the group consisting of hydroquinone, Bisphenol A, Bisphenol F, Bisphenol S, Bisphenol M, Bisphenol Z, Bisphenol AP.

7. The expandable, polymerizable composition according claim 1, wherein the ratio of benzoxazine equivalents to cyclic carbonate equivalents in the composition is from 99:1 to 1:99, preferably from 99:1 to 30:70.

8. The expandable, polymerizable composition according to claim 1, wherein the composition further comprises at least one reactive diluent.

9. The expandable, polymerizable composition according to claim 8, wherein the at least one reactive diluent is present at between 20% to 60% by weight of the benzoxazine.

10. The expandable, polymerizable composition according to claim 8, wherein the at least one reactive diluent is selected from the group consisting of 3-allyl-3,4-dihydro-2H-benzo[e][1,3]oxazine, 3-allyl-5-methyl-3,4-dihydro-2H-benzo[e][1,3]oxazine, 3-allyl-6-octyl-3,4-dihydro-2H-benzo[e][1,3]oxazine and 3-allyl-6-nonyl-3,4-dihydro-2H-benzo[e][1,3]oxazine.

11. A poly(benzoxazine)-co-poly(cyclic carbonate) polymerization product of the expandable, polymerizable composition according to claim 1.

12. The polymerization product according to claim 11, comprising a poly(benzoxazine)-co-poly(cyclic carbonate) of the general formula (V): 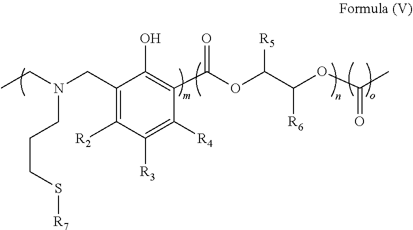
wherein:
R₂, R₃, and R₄ are each independently H, CH₃, C₂-C₁₅ straight, branched or cyclic alkyl optionally substituted with halogens, alkenyl or alkynyl groups, alkaryl groups, heteroalkyl groups, heteroaryl groups, hydroxy groups, disulfides, sulfonates, ether groups, thioether groups, ester groups, carboxylic acid groups, amine groups, amide groups, azides, or benzoxazine, and R₂ can be linked with R₃ to form a cyclic substituent on the benzene ring or R₃ can be linked with R₄ to form a cyclic substituent on the benzene ring, with the proviso that R₂, R₃, and/or R₄ comprises at least one unsaturated bond;
R₇ is C₂-C₁₅ straight, branched or cyclic alkyl optionally substituted with halogens, alkaryl groups, heteroalkyl groups, heteroaryl groups, hydroxy groups, disulfides, sulfonates, ether groups, thioether groups, ester groups, carboxylic acid groups, amine groups, amide groups and azides, with the proviso that R₇ comprises at least one thiol group;
R₅ and R₆ are each independently H, CH₃, C₂-C₁₅ straight, branched or cyclic alkyl optionally substituted with halogens, alkenyl, heteroalkyl groups and hydroxy groups;
m is an integer between 10 and 10,000;
n is an integer between 10 and 6,700; and
o is an integer between 0 and 1000.

13. A sealant, adhesive, coating, binding agent or dental filling comprising an expandable polymerizable composition according to claim 1.

14. Use of an expandable, polymerizable composition according to claim 1 as/in sealants, adhesives, coatings, binding agents or dental fillings.

15. A method of making a poly(benzoxazine)-co-poly(cyclic carbonate), the method comprising:
copolymerizing an expandable, polymerizable composition comprising at least one benzoxazine and at least one cyclic carbonate wherein the expandable, polymerizable composition shows volumetric expansion during the copolymerization upon a heat stimulus.

16. The method according to claim 15, wherein the benzoxazine is a compound of general Formula (I): 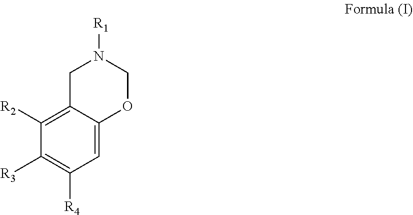
wherein R₁, R₂, R₃, and R₄ are each independently H, CH₃, C₂-C₁₅ straight, branched or cyclic alkyl optionally substituted with halogens, alkenyl or alkynyl groups, alkaryl groups, heteroalkyl groups, heteroaryl groups, hydroxy groups, disulfides, sulfonates, ether groups, thioether groups, ester groups, carboxylic acid groups, amine groups, amide groups, azides, or benzoxazines, and R₂ can be linked with R₃ to form a cyclic substituent on the benzene ring or R₃ can be linked with R₄ to form a cyclic substituent on the benzene ring.

17. The method according to claim 15, wherein the cyclic carbonate is a compound of general Formula (III): 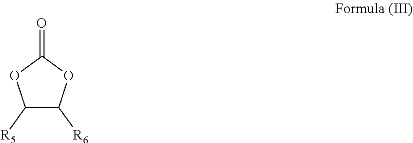
wherein R₅ and R₆ are each independently H, CH₃, C₂-C₁₅ straight, branched or cyclic alkyl optionally substituted with halogens, alkenyl, heteroalkyl groups and hydroxy groups.

18. The method according to claim 17, wherein the cyclic carbonate is ethylene carbonate or propylene carbonate.

19. The method according to claim 15, wherein the benzoxazine is derived from a dihydroxybenzene or a bisphenol, preferably selected from the group consisting of hydroquinone, Bisphenol A, Bisphenol F, Bisphenol S, Bisphenol M, Bisphenol Z, Bisphenol AP.

20. The method according to claim 15, wherein the ratio of benzoxazine equivalents to cyclic carbonate equivalents in the composition is from 99:1 to 1:99, preferably from 99:1 to 30:70.

21. The method according to claim 15, wherein the composition further comprises at least one reactive diluent.

22. The method according to claim 21, wherein the at least one reactive diluent is present at between 20% to 60% by weight of the benzoxazine.

23. The method according to claim 21, wherein the at least one reactive diluent is selected from the group consisting of 3-allyl-3,4-dihydro-2H-benzo[e][1,3]oxazine, 3-allyl-5-methyl-3,4-dihydro-2H-benzo[e][1,3]oxazine, 3-allyl-6-octyl-3,4-dihydro-2H-benzo[e][1,3]oxazine and 3-allyl-6-nonyl-3,4-dihydro-2H-benzo[e][1,3]oxazine.

24. The method according to claim 15 as/in sealants, adhesives, coatings, binding agents or dental fillings.

25. A poly(benzoxazine)-co-poly(cyclic carbonate) polymerization product obtained by co-polymerizing an expandable, polymerizable composition as defined in claim 15 at a temperature sufficient to initiate copolymerization.

26. The poly(benzoxazine)-co-poly(cyclic carbonate) polymerization product according to claim 25, comprising a poly(benzoxazine)-co-poly(cyclic carbonate) of the general formula (IV): 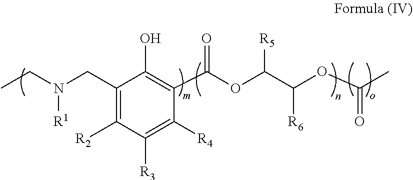
wherein:
R₁, R₂, R₃, and R₄ are each independently H, CH₃, C₂-C₁₅ straight, branched or cyclic alkyl optionally substituted with halogens, alkenyl or alkynyl groups, alkaryl groups, heteroalkyl groups, heteroaryl groups, hydroxy groups, disulfides, sulfonates, ether groups, thioether groups, ester groups, carboxylic acid groups, amine groups, amide groups, azides, or benzoxazine, and R₂ can be linked with R₃ to form a cyclic substituent on the benzene ring or R₃ can be linked with R₄ to form a cyclic substituent on the benzene ring;
R₅ and R₆ are each independently H, CH₃, C₂-C₁₅ straight, branched or cyclic alkyl optionally substituted with halogens, alkenyl, heteroalkyl groups and hydroxy groups;
m is an integer between 10 and 10,000;
n is an integer between 10 and 6,700; and
o is an integer between 0 and 1′000.

27. A process for manufacturing a poly(benzoxazine)-co-poly(cyclic carbonate) polymerization product, comprising the step of heating the expandable, polymerizable composition according to claim 1 to a temperature sufficient to initiate copolymerization.

28. The process according to claim 27, comprising the steps of:
providing the expandable, polymerizable composition according to claim 1, comprising crosslinkable benzoxazines and cyclic carbonates,
precuring the polymerizable composition by crosslinking the crosslinkable benzoxazines, and
heating the precured polymerizable composition to a temperature sufficient to initiate copolymerization of the crosslinked benzoxazines and cyclic carbonates.

29. A precured, expandable, polymerizable composition obtained by crosslinking crosslinkable benzoxazines comprised in an expandable polymerizable composition according to claim 1.

30. Use of a precured, expandable, polymerizable composition according to claim 29, as an expandable filling element for filling spaces in devices, wherein the precured, expandable composition has a pre-defined form, and wherein the precured, expandable composition shows volumetric expansion during copolymerization upon a heat stimulus.

31. Use of a precured, expandable, polymerizable composition according to claim 30 as a support and fixation element for windings of electrical machines or as a winding insulation barrier for electrical equipment.
